(19) United States
(12) Patent Application Publication
(10) Pub. No.: US 2025/0288624 A1
(43) Pub. Date: Sep. 18, 2025

(54) LONG-TERM STABILIZATION, FORMULATION AND TABLETING OF LIVE MICROBIAL CELLS

(71) Applicants: Massachusetts Institute of Technology, Cambridge, MA (US); The Brigham and Women`s Hospital, Inc., Boston, MA (US)

(72) Inventors: Robert S. Langer, Newton, MA (US); Carlo Giovanni Traverso, Newton, MA (US); Miguel Jimenez, Malden, MA (US); Johanna L`Heureux, Dudley, MA (US); Emily Kolaya, Cambridge, MA (US)

(73) Assignees: Massachusetts Institute of Technology, Cambridge, MA (US); The Brigham and Women`s Hospital, Inc., Boston, MA (US)

(21) Appl. No.: 18/477,970

(22) Filed: Sep. 29, 2023

Related U.S. Application Data
(60) Provisional application No. 63/428,708, filed on Nov. 29, 2022.

(51) Int. Cl. A61K 35/741; A61K 9/20; A61K 35/00; A61K 35/747; A61K 36/064

(52) U.S. Cl. CPC A61K 35/741; A61K 9/2018; A61K 35/747; A61K 36/064; A61K 2035/115

(57) ABSTRACT

Provided herein are compositions comprising Escherichia coli (E. coli), Saccharomyces boulardii (S. boulardii), Lactobacillus plantarum (L. plantarum), and/or Ensifer meliloti (E. meliloti) and a first stabilizing excipient. Also provided herein are methods of delivering E. coli, S. boulardii, L. plantarum, and/or E. meliloti to a subject in need thereof, methods of inducing bacterial growth in a subject or in a cell, tissue, or biological sample, and methods of inhibiting an enteric pathogen in a subject or in a cell, tissue, or biological sample. Further provided herein are methods of treating dysbiosis in a subject in need thereof.

### CROSS-REFERENCE TO RELATED APPLICATIONS

This application claims the benefit of and priority under 35 U.S.C. § 119(e) to U.S. Provisional Application No. 63/428,708, filed Nov. 29, 2022, titled LONG-TERM STABILIZATION, FORMULATION AND TABLETING OF LIVE MICROBIAL CELLS, the contents of which are incorporated herewith by reference in their entirety.

### GOVERNMENT SUPPORT

This invention was made with government support under NNX16A069A awarded by the National Aeronautics and Space Administration, and FA8650-21-2-7120 awarded by the Air Force Office of Scientific Research. The government has certain rights in the invention.

### SUMMARY

In one aspect, provided herein is composition comprising Escherichia coli (E. coli) and a first stabilizing excipient selected from enzymatic digest of soy (e.g., Bacto soytone), palatinose hydrate, D-(+)-turanose, maltitol, potassium gluconate, melibiose, sucrose, animal origin peptone derived from an infusion of beef (e.g., Bacto beef extract), L-rhamnose monohydrate, (+)-Sodium L-ascorbate, animal origin pancreatic digest of casein (e.g., Bacto tryptone), trehalose dihydrate, D-(+)-galactose, water-soluble portion of malted barley (e.g., Bacto malt extract), D-(+)-melezitose monohydrate, beta-lactose, D(−)-fructose, glucose, 1-kestose, or a combination thereof.

In another aspect, provided herein is a composition comprising Saccharomyces boulardii (S. boulardii) and a first stabilizing excipient selected from L-glutamic acid monosodium salt monohydrate, skim milk powder. D-(+)-turanose, water-soluble portion of malted barley (e.g., Bacto malt extract), maltitol, melibiose, lactulose, D-(+)-raffinose pentahydrate, palatinose hydrate, sucrose, animal origin pancreatic digest of casein (e.g., Bacto tryptone), glucose, enzymatic digest of soy (e.g., Bacto soytone), potassium gluconate, polydextrose, sodium gluconate, or a combination thereof.

In another aspect, provided herein is a composition comprising Lactobacillus plantarum (L. plantarum) and a first stabilizing excipient selected from pancreatic digest of gelatin (e.g., Gelysate peptone), water-soluble portion of malted barley (e.g., Bacto malt extract), animal origin pancreatic digest of casein (e.g., Bacto tryptone), sucrose, animal origin peptone derived from an infusion of beef (e.g., Bacto beef extract), D-sorbitol, polydextrose, trehalose dihydrate, L-glutamic acid monosodium salt monohydrate, beta-lactose, maltodextrin, L-rhamnose monohydrate, 1-kestose, animal-origin, enzymatic digest of bovine and porcine animal proteins (e.g., Bacto peptone), chondroitin sulfate A, or a combination thereof.

In another aspect, provided herein is a composition comprising Ensifer meliloti (E. meliloti) and a first stabilizing excipient selected from enzymatic digest of soy (e.g., Bacto soytone), skim milk powder, polydextrose, water-soluble portion of malted barley (e.g., Bacto malt extract), L-glutamic acid monosodium salt monohydrate, maltodextrin, palatinose hydrate, D-(+)-melezitose monohydrate, trehalose dihydrate, 1-kestose, maltose monohydrate, alpha-lactose monohydrate, sucrose, D-cellobiose, D-(+)-raffinose pentahydrate, melibiose, or a combination thereof.

The details of certain embodiments of the disclosure are set forth in the Detailed Description of Certain Embodiments, as described below. Other features, objects, and advantages of the disclosure will be apparent from the Definitions, Examples, and Claims.

### BRIEF DESCRIPTION OF THE DRAWINGS

FIG. 1. Survey of commercially available dry formulations of microbial probiotics. The viability of a wide representative range of commercial probiotic products was quantified (Table 1). Viable cell counts were defined as the number of colony forming units (CFUs) assessed through dilution plating on the appropriate solid medium and quantified via programmatic image analysis (see Example 8). Percent viable rates relative to promised cells were computed by dividing the CFU count by the CFU counts printed on the product label. Percent viable rates relative to total cells were computed by dividing the CFU counts by the number of total countable cells determined via automated fluorescence cytometry. N=4 or 5 independent dosage forms. Geometric means and geometric 95% confidence intervals plotted in black over individual replicates. Broad phylogenetic classes of the component microorganisms are noted in color and specific genera noted in italics (see FIGS. 5A-5B for detailed make up). neg.=negative; pos.=positive; Bifido.=Bifidobacterium; Lacto.=Lactobacillus; caps.=capsules.

FIGS. 2A-2E. Development and validation of high throughput pipeline for dry stabilized microbial materials. FIG. 2A. A high throughput pipeline (batch mixing, freeze-drying, plating and quantification) was developed to assess the capacity of a library of materials generally recognized as safe (G.R.A.S) to stabilize microorganisms (see Example 8). Illustration created with BioRender.com. FIG. 2B. Representative dried formulations made in high throughput and corresponding colony counting images. Scale bars=1 cm. FIG. 2C. Result of top 33% materials showing increased viability after drying across 4 organism and 2 concentrations and storage for 24 hours at room temperature, ordered by descending capacity to stabilize E. coli Nissle 1917 at the 5×concentration. The viability score is a composite colony count normalized to the maximum observable growth defined as 1 (see Example 8). See Table 2 for material identities and concentrations corresponding to the noted material indices and FIG. 8 for full results. FIG. 2D. Correlation analysis of top performing materials for each organism. FIG. 2E. Verification of top materials defined in FIG. 2C via a more precise assay (freeze drying in vials and individual CFU counts on plates) and the ability to preserve viability for 1 and 30 days at room temperature. Starred material indices are at 1×concentration, otherwise at 5×. Horizontal dashed line represents the limit of detection. Where noted by a vertical bar the water sample was assayed at higher concentrations to achieve a lower limit of detection. N=4-8 independently dried vials. Mean and SEM plotted over individual replicates.

FIGS. 3A-3E. Application of pipeline to make E. coli Nissle 1917 into a synthetic extremophile. FIG. 3A. The library of materials was assayed individually (X-axis) and in combination with melibiose (Y-axis) for its capacity to stabilize E. coli Nissle 1917 at room temperature and 50° C. for 24 hours. Black circles mark the mean viability score (see Example 8) along both dimensions and error bars represent the SEM. N=3. Colored shapes mark material combinations with melibiose selected for further characterization. Horizontal dashed line is a reference that marks the viability score of melibiose combined with vehicle (water, blue circle). Diagonal dashed line is the identity. All concentrations were at 1× as defined in Table 2. FOS: fructooligosacharides. FIG. 3B. The relative concentrations of each of the components in the selected two-material combinations (melibiose+{yeast extract or caffeine}) were varied, and the resulting effect on viability was measured after storage at 37° C. for 23 days. Concentrations are all relative to 1× as defined in Table 2. Letters “D” and “E” mark the selected formulations for further characterization. FIG. 3C. Direct stability comparison of formulation “D” (as noted in FIG. 3B), the parent formulation (melibiose), and capsules of the commercial E. coli Nissle 1917 product Mutaflor after storage at room temperature for 1 month. Mean and SEM plotted over individual replicates. N=3-5. ****, P<0.0001. FIG. 3D. Characterization of ultra-long stability at 37° C. compared to maltodextrin (the stabilizer used in Mutaflor). Mean and SEM plotted for each timepoint. N=3. Lower dashed line is the limit of detection. FIG. 3E. Transmission electron micrographs of E. coli Nissle 1917 cells after drying in formulation D or maltodextrin and optionally exposed to high temperature. Black arrowheads mark detached inner membranes.

FIGS. 4A-4H. Synthetic extremophiles withstand harsh processing to make microbial therapeutics for humans, agriculture, and space. FIG. 4A. Microbial tableting and coating allows simple dosing, transport and tuning of release to access a wide range of applications. FIG. 4B. Representative photos and scanning electron micrographs of synthetic extremophiles processed through the industrially relevant steps of milling, wet granulation, tableting and spray coating. FIG. 4C. Stability of the E. coli Nissle 1917 synthetic extremophile (Formulation D) through each process relative to the parent powder. Maltodextrin is the stabilizer used in the commercial product Mutaflor. Horizontal dotted line marks the limit of detection. Mean and SEM plotted over individual replicates. N=3. FIG. 4D. Tableting allows tuning of release kinetics through the inclusion of a matrix former (hydroxypropyl methylcellulose, HPMC). Synthetic extremophile E. coli Nissle 1917 cells carry a biosynthetic pathway (luxCDABE plasmid) and the released luminescence was tracked over time. Lines connect the means. Individual replicates plotted. N=2. FIG. 4E. High viability in the dry state allows higher resistance to ionizing radiation. Synthetic extremophile E. coli Nissle 1917 cells or a liquid suspension of the cells in PBS were exposed to the noted ionizing radiation dose. Lines connect the means. Individual replicates plotted. N=4. Two-way ANOVA: ****, P<0.0001. FIG. 4F. Both fresh E. coli Nissle 1917 and the synthetic extremophile (Formulation D) inhibit the enteric pathogen S. flexneri. The commercial comparator (maltodextrin) performs no better than a laboratory E. coli strain (DH5a). Mean and SEM plotted over individual replicates. N=3. One-way ANOVA: ***, P<0.001; **, P<0.01. FIG. 4G. E. meliloti is a nitrogen-fixing bacteria that supplies plants with nitrogen at symbiotic root nodules (inset). Synthetic extremophile E. meliloti was exposed to 50° C., hydrated and tested for functionality in a plant root nodulation assay with M. truncatula. Nodulated seedlings (arrows) can be quantified at day 12 post root inoculation. FIG. 4H. Quantification of nodulation assay (FIG. 4F) compared to the material viability score. Mean and SEM plotted over individual replicates. N=3-5 and 8 for controls (left of dashed line).

FIGS. 5A-5B. Raw CFU/g values of probiotics survey. FIG. 5A. Raw data used to calculate the percent viabilities graphed in FIG. 1. Means and standard errors of the mean (SEM) are plotted over individual replicates. FIG. 5B. Phylogenetic composition of the commercial products assessed. Numbers in boxes indicate the number of distinct strains in each clade. Full product details are summarized in Table 1.

FIGS. 6A-6B. Commercial probiotic product stress tested at 50° C. for 24 hours. FIG. 6A. Each product was kept at 4° C., 23° C. or 50° C. for 24 hours and the resulting viability was assessed (see Example 8). Only Mutaflor and VSL #3 were kept at 4° C. per manufacturer recommendations. FIG. 6B. Analysis of data in FIG. 6A. All viability values at 50° C. were divided by the mean viability at the manufacturer recommended storage temperature (4° C. for Mutaflor and VSL #3; 23° C. for rest of the products). Means and standard errors of the mean (SEM) are plotted over individual replicates for both panels.

FIGS. 7A-7C. High throughput pipeline for assessing viability score. FIG. 7A. Dried bacterial samples are rehydrated, diluted, plated onto 1-well plates in batch and incubated. The resulting array of spots is imaged, and each spot image (“raw”) is programmatically segmented (“mask”) into a region of interest (i.e., region containing bacterial colonies) and the segmented region is subdivided into countable “particles” using a watershed algorithm using Fiji. FIG. 7B. Additional image parameters from the segmented region such as the size relative to a control lawn size (spot are percent) and mean signal intensity relative to the region outside the segmented region (intensity over background) can be used to automatically classify spots into “lawns”, “blanks” and “countable” spots. The particle counts for blank spots are set to zero. The particle counts of countable spots are interpreted as the number of colonies. FIG. 7C. The particle counts of lawns are set to an organism specific value determined by extrapolating the line of best fit of all countable spots (i.e. those below grey region) to a particle count value corresponding to a spot area percent of 120%.

FIG. 8. Detailed results of material stabilizer library. Extended data of FIG. 2C plotted with a continuous color scale. Grey color denotes zero observed viability. Vertical black boxes mark the positive controls (#18: ATCC reagent 20 and #35: trehalose) and vertical red boxes mark the negative controls (#224: sodium hydroxide and #238: sodium metabisulfite). Viability score is a composite score of the colony counts across three plating dilutions (see Example 8). “Rel. mat. conc.”—relative material concentrations as defined in Table 2 for 5× and 1×.

FIG. 9. Compound classes over-represented in hit materials. All compounds were classified by chemical structure. Top performing compounds are the same ones included in the correlation analysis (FIG. 2D, see Example 8). Extract refers to cell or plant extracts such as yeast extract or malt extract. Peptone refers to both protein digests (tryptone) or other protein-based materials (catalase). Mixtures of individually prepared components were omitted from this analysis.

FIG. 10. Validation of two-material formulations at 37° C. Materials selected from the two-material library were validated at a larger scale (see Example 8) to determine the precise retention of viability when stored at 37° C. for the specified time. “Powders alone” refers to storage of the freeze-dried and milled powders in nitrogen-flushed bags with desiccant. “Powders+excipients” refers to storage of the milled microbial powders mixed with excipients (binder, filler, glidant) as described in Example 8. Means and standard errors of the mean (SEM) are plotted over individual replicates for both panels.

FIG. 11. Individual viability traces of top materials formulations at different storage temperatures. Extended data from FIG. 2D. Freeze-dried microbial powders were stored after milling and mixing with excipients (see Example 8). Storage was in glass vials with desiccant in the dark at the specified temperatures. Lines connect the geometric means and error bars mark the 95% confidence intervals. N=3.

FIG. 12. Preculture medium impacts ultimate survival through lyophilization. E. coli Nissle 1917 was grown in a range of preculture media, washed in PBS, mixed with trehalose, freeze-dried, hydrated, inoculated in fresh LB and then mixed with Presto Blue to assess the viability. Values represent the fold increase in Presto Blue signal relative to the control medium (LB). Each medium has three components a Peptone (P #) at 10 g/L, an Extract (E #) at 5 g/L and a Salt (NaCl) concentration (S #). P1: Tryptone, P2: Soytone, P3: Gelysate peptone, P4: Bacto peptone, E1: Beef extract, E2: Yeast extract, E3: Malt extract, E4: Beef & yeast extract, S0: 86 mM NaCl, S1: 250 mM NaCl, S2: 600 mM NaCl. The control LB well is P1:E2:S0.

FIG. 13. Time of bacterial harvest impacts ultimate survival through lyophilization. Liquid flask cultures of E. coli Nissle 1917 were harvested at the specified times since inoculation. The viability of the culture at the time of harvest was assessed by plating and a percent viability was calculated by dividing by the estimated CFU/mL estimated from the optical density measurement (1 OD600˜1E9 CFU/mL). Each sample of harvested cells was freeze-dried and the resulting viability was assessed after 24 hours of storage at 23° C. (see Example 8). Lines connect the means and error bars mark the standard error of the means. N=2.

FIG. 14. Bacterial loading can be increased in powders and tablets. E. coli Nissle 1917 was freeze dried with Formulation D at three different concentrations of the initial cell suspension. The resulting powders were milled and mixed with excipients (see Example 8) and stored as mixed (right) or tableted and then stored (left). All samples were stored in nitrogen-flushed bags with desiccant at 37° C. for the specified time. Means and standard errors of the mean (SEM) are plotted over individual replicates.

FIG. 15. Combination of preculture timing and increased bacterial loading leads to viabilities above 10¹⁰ CFU per gram. Either the commercial product Mutaflor or the synthetic extremophile E. coli Nissle 1917 (melibiose 1×) was evaluated for their initial viability and viability after long-term storage at 23° C. for 32 days. “100×” refers to a 100-fold increase in the bacterial concentration of the bacterial suspension mixed with the stabilizer. “ideal time” refers to harvesting the bacterial flask culture at the optimal time of 9 hours as indicated by the data in FIG. 14. Geometric means and 95% confidence intervals are plotted over individual replicates.

FIG. 16. Tableting pressure modulates stability of bacteria at 37° C. E. coli Nissle 1917 was freeze dried with Formulation D at a 10×cell concentration of the initial bacterial suspension. The resulting material was milled, mixed with excipients, and pressed into tablets (see Example 8) at the pressure indicated. Means and standard errors of the mean (SEM) are plotted over individual replicates.

FIG. 17. Synthetic extremophile E. coli Nissle 1917 extremophile survives in SGF for 1 hour. Extended data from FIG. 4C. Synthetic extremophile E. coli Nissle 1917 (Formulation D) or the commercial comparator (freeze-dried with 5% maltodextrin) were milled, tableted and coated with Eudragit S100 (see Example 8). The coated tablets were subsequently submerged in simulated gastric fluid (SGF) for 1 hour at 37° C. to simulate ingestion and passage through the stomach. The viability of each sample was assessed by plating. Viability is normalized to the viability of the milled powder. Coated tablets were cut in half immediately before rehydration to expose inner contents. Geometric means and 95% confidence intervals are plotted over individual replicates. Horizontal dotted line marks the limit of detection.

FIG. 18. E. meliloti stabilizers against 24 hours at 50° C. Extended data of FIG. 8. E. meliloti was mixed with the material library at the 1×concentration, freeze dried, and exposed to 50° C. for 24 hours. The viability score was characterized as in FIG. 8, and the data is ordered by the viability score of the comparison values when stored at 23° C.

FIGS. 19A-19D. Characterization of E. coli Nissle 1917 cell envelope ultrastructure after drying and rehydration. FIG. 19A. Representative transmission electron micrographs of dried and rehydrated bacterial cells. Cells were either dried in Formulation D or the commercial comparator (maltodextrin) and optionally exposed to 50° C. (see Example 8). FIG. 19B. Enlarged view of areas marked in dashed rectangles from FIG. 19A. Additional dashed rectangles mark areas included in FIG. 3E. FIG. 19C. The objects in three image fields for each of the four conditions were classified into the three noted categories and quantified (see Example 8). N=3 image fields. Error bars represent the standard error of the mean (SEM). Insets show multiple representative objects in each class. Inset field of view is 1 um×1 um. FIG. 19D. Analysis of data in FIG. 19C. The number of cells with detached inner membranes was divided by the total number of cells (excluding cell debris). Ordinary one-way ANOVA: ****, P<0.0001.

FIGS. 20A-20B. Inhibition of the enteric pathogen Shigella flexneri by E. coli Nissle 1917 is not impacted by stabilizers materials. FIG. 20A. S. flexneri was cultured either with fresh E. coli Nissle 1917 cells or mixed with a vehicle control (water), the materials used to make formulation D (“D materials”) or maltodextrin. Shigella inhibition was quantified by qPCR (see Example 8). N=3. Ordinary one-way ANOVA: ns, P>0.05. FIG. 20B. S. flexneri was cultured with a vehicle control (water) or co-cultured with fresh E. coli Nissle 1917 cells either alone or as a mixture with the materials used to make formulation D (“D materials”) or maltodextrin. Shigella inhibition was quantified by qPCR (see Example 8). N=3. Ordinary one-way ANOVA: ns, P>0.05.

### DEFINITIONS

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the stomach and/or intestines. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severeity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating dysbiosis.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for preventing dysbiosis.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

The term “polymer” refers to a compound comprising eleven or more covalently connected repeating units. In certain embodiments, a polymer is naturally occurring. In certain embodiments, a polymer is synthetic (i.e., not naturally occurring).

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles, macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a nanoparticle. In certain embodiments, the particle is a microparticle.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compositions

Escherichia coli

In one aspect, provided herein is a composition comprising Escherichia coli (E. coli) and a first stabilizing excipient selected from enzymatic digest of soy (e.g., Bacto soytone), palatinose hydrate, D-(+)-turanose, maltitol, potassium gluconate, melibiose, sucrose, animal origin peptone derived from an infusion of beef (e.g., Bacto beef extract), L-rhamnose monohydrate, (+)-Sodium L-ascorbate, animal origin pancreatic digest of casein (e.g., Bacto tryptone), trehalose dihydrate, D-(+)-galactose, water-soluble portion of malted barley (e.g., Bacto malt extract), D-(+)-melezitose monohydrate, beta-lactose, D(−)-fructose, glucose, 1-kestose, or a combination thereof.

In certain embodiments, the first stabilizing excipient is selected from enzymatic digest of soy (e.g., Bacto soytone), palatinose hydrate, (D)-(+)-turanose, maltitol, melibiose, (+)-sodium L-ascorbate, trehalose dihydrate, beta-lactose, glucose, potassium gluconate, or a combination thereof. In certain embodiments, the first stabilizing excipient is selected from enzymatic digest of soy (e.g., Bacto soytone), palatinose hydrate, (D)-(+)-turanose, maltitol, melibiose, (+)-sodium L-ascorbate, trehalose dihydrate, beta-lactose, glucose, or a combination thereof. In certain embodiments, the first stabilizing excipient is selected from enzymatic digest of soy (e.g., Bacto soytone), palatinose hydrate, (D)-(+)-turanose, maltitol, melibiose, (+)-sodium L-ascorbate, trehalose dihydrate, beta-lactose, glucose, or a combination thereof. In certain embodiments, the first stabilizing excipient is melibiose.

In certain embodiments, the composition comprises a second stabilizing excipient selected from short inulin, concentrates of the water-soluble portion of autolyzed Saccharomyces cerevisiae cells (e.g., Bacto yeast extract), animal origin pancreatic digest of casein (e.g., Bacto tryptone), porcine mucin type III, 1,4-benzenedimethanol, caffeine, enzymatic digest of soy (e.g., Bacto soytone), sucralose, dioctyl-sulfosuccinate, 2-methyl-1-propanol, propyl gallate, β-glycerophosphate disodium salt hydrate, DL-β-(2-thienyl)serine, melibiose, animal origin peptone derived from an infusion of beef (e.g., Bacto beef extract), sodium citrate dihydrate, N-phenylthiourea, 4-guanidinobutyric acid, calcium D-gluconate, or a combination thereof. In certain embodiments, the second stabilizing excipient is selected from short inulin, concentrates of the water-soluble portion of autolyzed Saccharomyces cerevisiae cells (e.g., Bacto yeast extract), caffeine, or a combination thereof. In certain embodiments, the second stabilizing excipient is selected from concentrates of the water-soluble portion of autolyzed Saccharomyces cerevisiae cells (e.g., Bacto yeast extract), caffeine, or a combination thereof.

In certain embodiments, the first stabilizing excipient is melibiose, and the second stabilizing excipient is concentrates of the water-soluble portion of autolyzed Saccharomyces cerevisiae cells (e.g., Bacto yeast extract) or caffeine. In certain embodiments, the first stabilizing excipient is present in a concentration of between about 7% and about 50% (w/w). In certain embodiments, the first stabilizing excipient is melibiose present in a concentration of between about 0.3% and about 9% (w/w). In certain embodiments, the second stabilizing excipient is present in a concentration of between about 0.01% and about 7% (w/w).

In certain embodiments, the second stabilizing excipient is concentrates of the water-soluble portion of autolyzed Saccharomyces cerevisiae cells (e.g., Bacto yeast extract) present in a concentration of between about 0.04% and about 7% (w/w). In certain embodiments, the first stabilizing excipient is melibiose present in a concentration of about 1% (w/w), and the second stabilizing excipient is concentrates of the water-soluble portion of autolyzed Saccharomyces cerevisiae cells (e.g., Bacto yeast extract) present in a concentration of about 0.04% (w/w) or about 5% (w/w).

In certain embodiments, the second stabilizing excipient is caffeine present in a concentration of between about 0.01% and about 0.4% (w/w). In certain embodiments, the first stabilizing excipient is melibiose present in a concentration of about 1% (w/w), and the second stabilizing excipient is caffeine present in a concentration of about 0.01% (w/w).

In certain embodiments, the E. coli is E. coli Nissle 1917.

Saccharomyces boulardii

In another aspect, provided herein is a composition comprising Saccharomyces boulardii (S. boulardii) and a first stabilizing excipient selected from L-glutamic acid monosodium salt monohydrate, skim milk powder. D-(+)-turanose, water-soluble portion of malted barley (e.g., Bacto malt extract), maltitol, melibiose, lactulose, D-(+)-raffinose pentahydrate, palatinose hydrate, sucrose, animal origin pancreatic digest of casein (e.g., Bacto tryptone), glucose, enzymatic digest of soy (e.g., Bacto soytone), potassium gluconate, polydextrose, sodium gluconate, or a combination thereof.

In certain embodiments, the first stabilizing excipient is selected from L-glutamic acid monosodium salt monohydrate, skim milk powder, D-(+)-Turanose, water-soluble portion of malted barley (e.g., Bacto malt extract), maltitol, melibiose, D-(+)-raffinose pentahydrate, palatinose hydrate, sucrose, animal origin pancreatic digest of casein (e.g., Bacto tryptone), glucose, potassium gluconate, or a combination thereof. In certain embodiments, first stabilizing excipient is selected from water-soluble portion of malted barley (e.g., Bacto malt extract), D-(+)-turanose, D-(+)-raffinose pentahydrate, L-glutamic acid monosodium salt monohydrate, maltitol, palatinose hydrate, skim milk powder, melibiose, sucrose, glucose, animal origin pancreatic digest of casein (e.g., Bacto tryptone), or a combination thereof.

Lactobacillus plantarum

In another aspect, provided herein is a composition comprising Lactobacillus plantarum (L. plantarum) and a first stabilizing excipient selected from pancreatic digest of gelatin (e.g., Gelysate peptone), water-soluble portion of malted barley (e.g., Bacto malt extract), animal origin pancreatic digest of casein (e.g., Bacto tryptone), sucrose, animal origin peptone derived from an infusion of beef (e.g., Bacto beef extract), D-sorbitol, polydextrose, trehalose dihydrate, L-glutamic acid monosodium salt monohydrate, beta-lactose, maltodextrin, L-rhamnose monohydrate, 1-kestose, animal-origin, enzymatic digest of bovine and porcine animal proteins (e.g., Bacto peptone), chondroitin sulfate A, or a combination thereof.

In certain embodiments, the first stabilizing excipient is selected from water-soluble portion of malted barley (e.g., Bacto malt extract), pancreatic digest of gelatin (e.g., Gelysate peptone), animal origin pancreatic digest of casein (e.g., Bacto tryptone), sucrose, animal origin peptone derived from an infusion of beef (e.g., Bacto beef extract), D-sorbitol, or a combination thereof. In certain embodiments, the first stabilizing excipient is selected from animal origin peptone derived from an infusion of beef (e.g., Bacto beef extract), sucrose, pancreatic digest of gelatin (e.g., Gelysate peptone), D-sorbitol, animal origin pancreatic digest of casein (e.g., Bacto tryptone), or a combination thereof.

Ensifer meliloti

In another aspect, provided herein is a composition comprising Ensifer meliloti (E. meliloti) and a first stabilizing excipient selected from enzymatic digest of soy (e.g., Bacto soytone), skim milk powder, polydextrose, water-soluble portion of malted barley (e.g., Bacto malt extract), L-glutamic acid monosodium salt monohydrate, maltodextrin, palatinose hydrate, D-(+)-melezitose monohydrate, trehalose dihydrate, 1-kestose, maltose monohydrate, alpha-lactose monohydrate, sucrose, D-cellobiose, D-(+)-raffinose pentahydrate, melibiose, or a combination thereof.

In certain embodiments, the first stabilizing excipient is selected from enzymatic digest of soy (e.g., Bacto soytone), skim milk powder, polydextrose, water-soluble portion of malted barley (e.g., Bacto malt extract), L-glutamic acid monosodium salt monohydrate, maltodextrin, palatinose hydrate, D-(+)-melezitose monohydrate, or a combination thereof.

In certain embodiments, the first stabilizing excipient is selected from enzymatic digest of soy (e.g., Bacto soytone), skim milk powder, polydextrose, water-soluble portion of malted barley (e.g., Bacto malt extract), L-glutamic acid monosodium salt monohydrate, or a combination thereof. In certain embodiments, the first stabilizing excipient is selected from enzymatic digest of soy (e.g., Bacto soytone), skim milk powder, L-glutamic acid monosodium salt monohydrate, or a combination thereof. In certain embodiments, the first stabilizing excipient is selected from trehalose dihydrate, 1-kestose, palatinose hydrate, maltose monohydrate, alpha-lactose monohydrate, enzymatic digest of soy (e.g., Bacto soytone), D-(+)-melezitose monohydrate, sucrose, D-cellobiose, D-(+)-raffinose pentahydrate, melibiose, polydextrose, maltodextrin, or a combination thereof. In certain embodiments, the first stabilizing excipient is selected from trehalose dihydrate, 1-kestose, palatinose hydrate, maltose monohydrate, alpha-lactose monohydrate, enzymatic digest of soy (e.g., Bacto soytone), D-(+)-melezitose monohydrate, sucrose, D-cellobiose, D-(+)-raffinose pentahydrate, melibiose, or a combination thereof. In certain embodiments, the first stabilizing excipient is selected from sucrose, maltose monohydrate, polydextrose, maltodextrin, enzymatic digest of soy (e.g., Bacto soytone), or a combination thereof. In certain embodiments, the first stabilizing excipient is selected from sucrose, maltose monohydrate, polydextrose, or a combination thereof. In certain embodiments, E. meliloti is equivalent to Sinorhizobium meliloti (S. meliloti).

### Additional Embodiments

In certain embodiments, the composition is any composition disclosed herein.

In certain embodiments, the composition exhibits at least about 1×10⁶, at least about 1×10⁷, at least about 1×10⁸, at least about 1×10⁹, or at least about 1×10¹⁰ colony forming units (CFU)/gram after 30 days at 23° C.

In certain embodiments, the composition is stable at about 23° C., about 37° C., or about 50° C. for at least 1 day. In certain embodiments, the composition is stable at about 23° C., about 37° C., or about 50° C. for at least 1 month. In certain embodiments, the composition is stable at less than about 5% relative humidity for at least 1 day. In certain embodiments, the composition is stable at less than about 5% relative humidity for at least 1 month. In certain embodiments, the composition is stable at about 23° C., about 37° C., or about 50° C., and at less than about 5% relative humidity, for at least 1 day. In certain embodiments, the composition is stable at about 23° C., about 37° C., or about 50° C., at less than about 5% relative humidity, for at least 1 month. In certain embodiments, the composition retains at least about 10% viability at about 23° C., about 37° C., or about 50° C. for at least about 6.5 months.

In certain embodiments, the composition is stable upon exposure to an organic solvent. In certain embodiments, the organic solvent is acetone or isopropanol.

In certain embodiments, the composition retains at least about 5%, at least about 10%, or at least about 15% viability upon milling. In certain embodiments, the milling comprises exposure to shear forces.

In certain embodiments, the composition retains at least about 5%, at least about 10%, or at least about 15% viability upon tableting. In certain embodiments, the tableting comprises application of about 3.4 kN of force per tablet. In certain embodiments, the tableting comprises application of up to about 6 kN of force per tablet. In certain embodiments, the tableting comprises exposure to an increased pressure. In certain embodiments, the increased pressure is about 173 uPa per tablet. In certain embodiments, the increased pressure is up to about 650 uPa per tablet.

In certain embodiments, the composition retains at least about 20% or at least about 28% viability upon wet granulation. In certain embodiments, the wet granulation comprises exposure to an organic solvent and baking. In certain embodiments, the organic solvent is isopropanol. In certain embodiments, the baking comprises exposure to a temperature of at least about 45° C.

In certain embodiments, the composition retains at least about 0.2%, at least about 1%, or at least about 3% viability upon spray coating. In certain embodiments, the spray coating comprises exposure to an organic solvent and a polymer, and baking. In certain embodiments, the organic solvent is acetone and/or isopropanol. In certain embodiments, the polymer is an enteric polymer. In certain embodiments, the enteric polymer is Eudragit L100-55, Eudragit L100, Eudragit S100, or a combination thereof. In certain embodiments, the baking comprises exposure to a temperature of at least about 40° C.

In certain embodiments, the composition is stable upon exposure to acid. In certain embodiments, the composition is stable after exposure to a solution of about pH 1.2 for at least about 1 hour at about 37° C. In certain embodiments, the composition retains at least about 90% or at least about 95% viability after exposure to a solution of about pH 1.2 for at least about 1 hour at about 37° C.

In certain embodiments, the composition is stable upon exposure to ionizing radiation. In certain embodiments, the ionizing radiation is at least about 0.01 kGy, at least about 0.1 kGy, or at least about 1 kGy.

In certain embodiments, the composition is stable upon exposure to ionizing radiation in space. In certain embodiments, the composition is stable upon exposure to ionizing radiation in space between Earth and Mars. In certain embodiments, the composition is stable upon exposure to ionizing radiation on the surface of Earth. In certain embodiments, the composition is stable upon exposure to ionizing radiation on the surface of Mars. In certain embodiments, the composition is stable upon exposure to ionizing radiation in space between Earth and the International Space Station (ISS). In certain embodiments, the composition is stable upon exposure to ionizing radiation on the outside of the ISS.

In certain embodiments, the composition is capable of inducing bacterial growth in a plant. In certain embodiments, the inducing bacterial growth comprises inducing nodulation in the plant.

In certain embodiments, the composition is capable of inhibiting an enteric pathogen. In certain embodiments, the enteric pathogen is Shigella flexneri.

In certain embodiments, the composition comprises a solid dosage form. In certain embodiments, the solid dosage form is a tablet. In certain embodiments, the solid dosage form comprises an immediate release layer. In certain embodiments, the solid dosage form comprises a slow release layer. In certain embodiments, the slow release layer comprises a matrix forming polymer. In certain embodiments, the matrix forming polymer is hydroxypropyl methylcellulose (HPMC).

In certain embodiments, the composition further comprises an additional microorganism.

In certain embodiments, the E. coli, S. boulardii, L. plantarum, or E. meliloti is precultured in a medium comprising a peptone, an extract, and a salt concentration. In certain embodiments, the peptone is animal origin pancreatic digest of casein (e.g., Bacto tryptone), enzymatic digest of soy (e.g., Bacto soytone), pancreatic digest of gelatin (e.g., Gelysate peptone), animal-origin, enzymatic digest of bovine and porcine animal proteins (e.g., Bacto peptone), or a combination thereof. In certain embodiments, the extract is animal origin peptone derived from an infusion of beef (e.g., Bacto beef extract), concentrates of the water-soluble portion of autolyzed Saccharomyces cerevisiae cells (e.g., Bacto yeast extract), water-soluble portion of malted barley (e.g., Bacto malt extract), or a combination thereof. In certain embodiments, the salt concentration is between about 50 mM and about 800 mM NaCl.

### Methods of Delivering Microorganisms, Inducing Bacterial Growth, Inhibiting an Enteric Pathogen, and Treatment

Provided herein are methods of delivering microorganisms, inducing bacterial growth, inhibiting an enteric pathogen, and treatment. Said methods comprise administering a “composition” or a “provided composition,” wherein said composition or provided composition is any composition disclosed herein.

In another aspect, provided herein is a method of delivering Escherichia coli (E. coli) to a subject in need thereof, comprising administering to the subject a provided composition. In certain embodiments, the method of delivering E. coli comprises administering to the subject a composition comprising E. coli and a first stabilizing excipient selected from enzymatic digest of soy (e.g., Bacto soytone), palatinose hydrate, D-(+)-turanose, maltitol, potassium gluconate, melibiose, sucrose, animal origin peptone derived from an infusion of beef (e.g., Bacto beef extract), L-rhamnose monohydrate, (+)-Sodium L-ascorbate, animal origin pancreatic digest of casein (e.g., Bacto tryptone), trehalose dihydrate, D-(+)-galactose, water-soluble portion of malted barley (e.g., Bacto malt extract), D-(+)-melezitose monohydrate, beta-lactose, D(−)-fructose, glucose, 1-kestose, or a combination thereof.

In another aspect, provided herein is a method of delivering Saccharomyces boulardii (S. boulardii) to a subject in need thereof, comprising administering to the subject a provided composition. In certain embodiments, the method of delivering S. boulardii comprises administering to the subject a composition comprising S. boulardii and a first stabilizing excipient selected from L-glutamic acid monosodium salt monohydrate, skim milk powder. D-(+)-turanose, water-soluble portion of malted barley (e.g., Bacto malt extract), maltitol, melibiose, lactulose, D-(+)-raffinose pentahydrate, palatinose hydrate, sucrose, animal origin pancreatic digest of casein (e.g., Bacto tryptone), glucose, enzymatic digest of soy (e.g., Bacto soytone), potassium gluconate, polydextrose, sodium gluconate, or a combination thereof.

In another aspect, provided herein is a method of delivering Lactobacillus plantarum (L. plantarum) to a subject in need thereof, comprising administering to the subject a provided composition. In certain embodiments, the method of delivering L. plantarum comprises administering to the subject a composition comprising L. plantarum and a first stabilizing excipient selected from pancreatic digest of gelatin (e.g., Gelysate peptone), water-soluble portion of malted barley (e.g., Bacto malt extract), animal origin pancreatic digest of casein (e.g., Bacto tryptone), sucrose, animal origin peptone derived from an infusion of beef (e.g., Bacto beef extract), D-sorbitol, polydextrose, trehalose dihydrate, L-glutamic acid monosodium salt monohydrate, beta-lactose, maltodextrin, L-rhamnose monohydrate, 1-kestose, animal-origin, enzymatic digest of bovine and porcine animal proteins (e.g., Bacto peptone), chondroitin sulfate A, or a combination thereof.

In another aspect, provided herein is a method of delivering Ensifer meliloti (E. meliloti) to a subject in need thereof, comprising administering to the subject a provided composition. In certain embodiments, the method of delivering E. meliloti comprises administering to the subject a composition comprising E. meliloti and a first stabilizing excipient selected from enzymatic digest of soy (e.g., Bacto soytone), skim milk powder, polydextrose, water-soluble portion of malted barley (e.g., Bacto malt extract), L-glutamic acid monosodium salt monohydrate, maltodextrin, palatinose hydrate, D-(+)-melezitose monohydrate, trehalose dihydrate, 1-kestose, maltose monohydrate, alpha-lactose monohydrate, sucrose, D-cellobiose, D-(+)-raffinose pentahydrate, melibiose, or a combination thereof.

In another aspect, provided herein is a method of inducing bacterial growth in a subject or in a cell, tissue, or biological sample, comprising administering to the subject or contacting the cell, tissue, or biological sample with a provided composition. In certain embodiments, the cell, tissue, or biological sample is a plant. In certain embodiments, the method comprises inducing nodulation in the plant.

In another aspect, provided herein is a method of inhibiting an enteric pathogen in a subject or in a cell, tissue, or biological sample, comprising administering to the subject or contacting the cell, tissue, or biological sample with a provided composition. In certain embodiments, the enteric pathogen is Shigella flexneri.

In another aspect, provided herein is a method of treating dysbiosis in a subject in need thereof, comprising administering to the subject a provided composition. In certain embodiments, the subject in need thereof has a gastrointestinal disorder.

### EXAMPLES

### Example 1

Microorganisms have been central to human technological progress and continue to be key in wide ranging fields from food production (e.g., baked goods) to biologics manufacturing (e.g., synthetic insulin)¹. However, by and large, microbial cells are kept alive only during the manufacturing process and are destroyed, deactivated, or removed from the final product. However, through the advent of culture-independent sequencing techniques and synthetic biology, the pharmaceutical, agricultural and space health fields have now turned to developing live microorganisms as the final product to cure disease², enhance crop production³ and for on-demand bioproduction⁴.

Of importance to these new microbial technologies is maintenance of high cell viability throughout the entire life cycle of the product. To achieve this, researchers and commercial companies have turned to microbial cryopreservation methods⁵ or to organisms that have natural extremophilic properties (e.g., dehydration-resistance, acid-resistance)⁶. However, the need for select organisms or costly, inflexible cold chains severely limits the use case of live microbial products.

An alternative solution would be dry, shelf-stable microbial materials that are simple to package, ship, and use. While there has been extensive research on the lyopreservation of microorganisms, these previous studies have primarily focused on storage of microorganisms in culture collections which only require “acceptable” levels of viability (i.e., just enough viable cells for recovery through culture amplification)¹⁰. Furthermore, while these studies have defined sugars and peptides with high glass transition temperatures as generally good stabilizers, these studies have also shown that specific viability results vary widely depending on the microorganism in question¹⁰.

Therefore, examples of applications using this lyopreservation knowledge were investigated, with the aim of generating the high viabilities that would be required for a functional microbial product. Available commercial examples of dried microbial products (i.e., probiotics) were evaluated to understand the current state of microbial stabilization. Generally low viabilities were found, with particularly poor results for gram-negative bacteria (e.g., E. coli). To fill this gap, material-based synthetic extremophiles were developed that are shelf-stable without refrigeration. These stabilized microorganisms can withstand the extreme conditions encountered in pharmaceutical manufacturing pipelines (e.g., heat, pressure, solvents). This stabilization approach maintains the functional capacity of the microorganisms in both a bioluminescence and plant root nodulation assay.

### Example 2: Survey of Commercial Probiotic Products

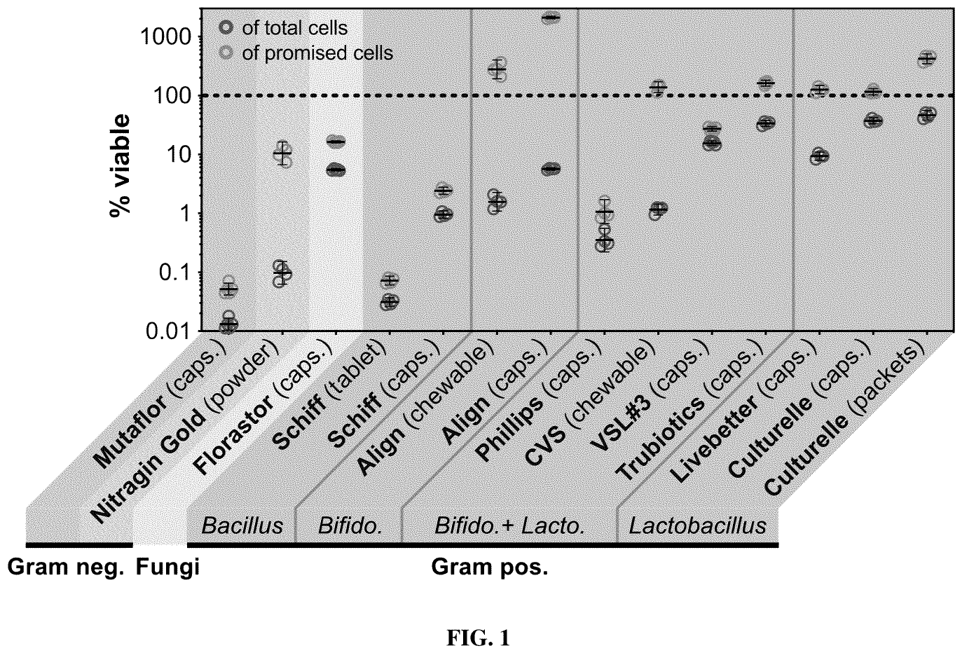

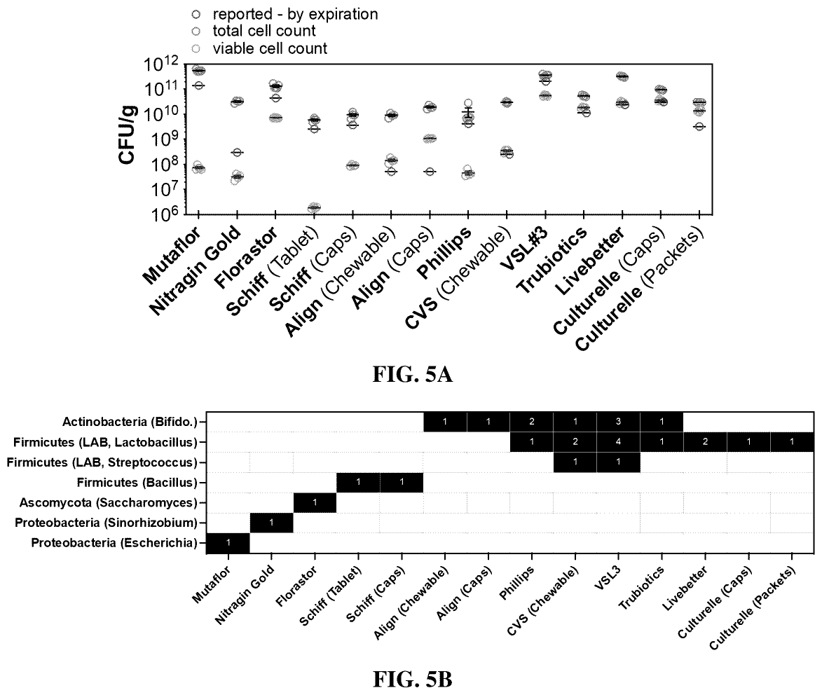

A salient example of dried microbial products are the current commercially available probiotics marketed for human use. These products represent the current state of the art of microbial stabilization for commercial applications. However, when the viable cell counts (colony forming units, CFUs) were surveyed across a range of off-the-shelf probiotics (Table 1), only 7 in 13 products were found to contain viable cell counts at or higher than the promised amount on the label (FIG. 1, FIGS. 5A-5B), with a mean (geo.) viability of just ˜21% of that promised. Nevertheless, when the total cell count was assessed microscopically (both dead and alive cells), all products contained cell totals above those promised (FIG. 5A), suggesting a loss of viability during and following manufacture. When the viable cell count was compared to this microscopically determined total cell count, only 1 in 13 products had a viability greater than 40%, with 6 in 13 products with viabilities lower than 2% and an overall mean viability of just 1.9% of total cells (FIG. 1).

Beyond this overall low viability rate, a lack of variety was also found in terms of microbial species identity (FIG. 5B). A majority (9 of 13) of products used microorganisms from just two groups (Lactobacillus, Bifidobacterium) which encompassed all the products with higher viabilities (FIG. 5B). There was only one commercially available representative in the large clade of gram-negative bacteria (Mutaflor, Escherichia coli Nissle 1917) and it had the lowest viability (0.05% of promised and 0.01% of total cells). To corroborate this finding, one additional commercial gram-negative product marketed for plant-use (Nitragin Gold, Ensifer meliloti) was tested and was found to also have a low viability (10% of promised and 1% of total cells) (FIG. 1).

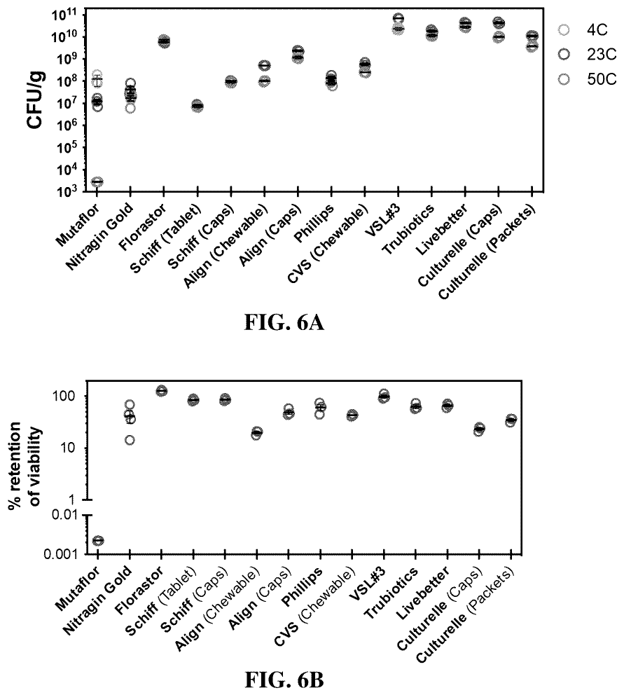

Furthermore, to evaluate the intrinsic extremophilic qualities, these products were stress tested by exposure to a temperature of 50° C. for 24 hours. Nearly all the products were found to retain an acceptable viability of >10% relative to non-stressed samples (FIGS. 6A-6B). However, the E. coli Nissle 1917 product Mutaflor retained only 0.002% viability (FIGS. 6A-6B). Overall, the above survey points to an industry consolidated around a small number of gram-positive microorganisms that are easy to stabilize and poor options for gram-negative organisms like E. coli Nissle 1917. In contrast, the synthetic biology and microbial therapeutics research fields in academia are consolidated around gram-negative bacteria, with many of the most-forward looking examples (e.g., cancer treatment) using specifically E. coli Nissle 1917^{2,7,8}.

### Example 3: High Throughput Pipeline for Generating Synthetic Extremophiles

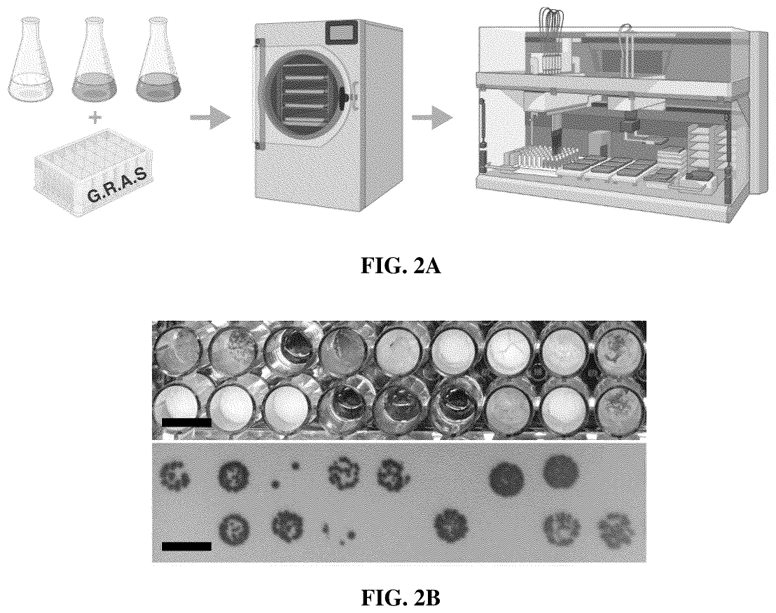

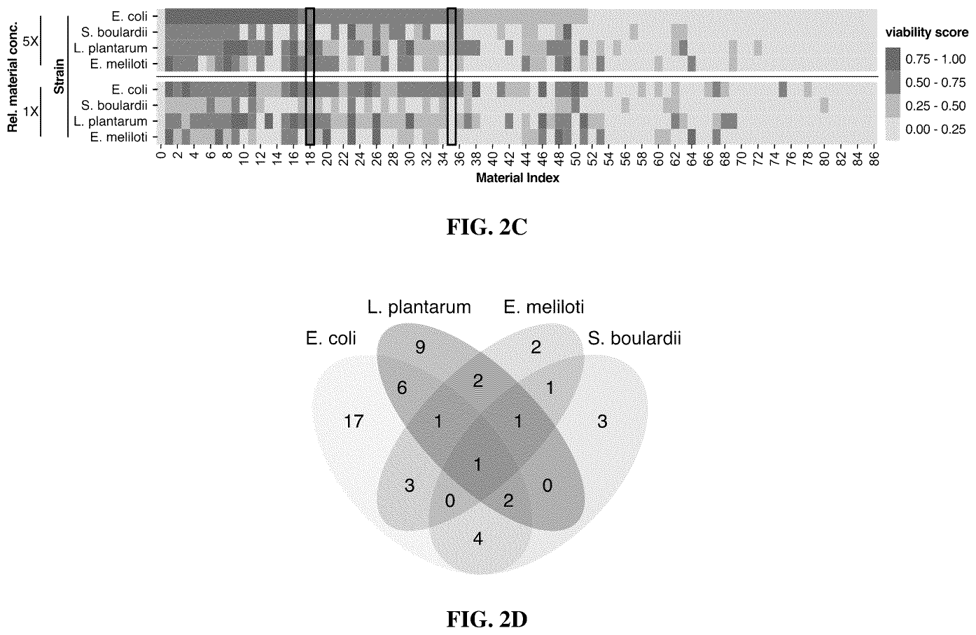

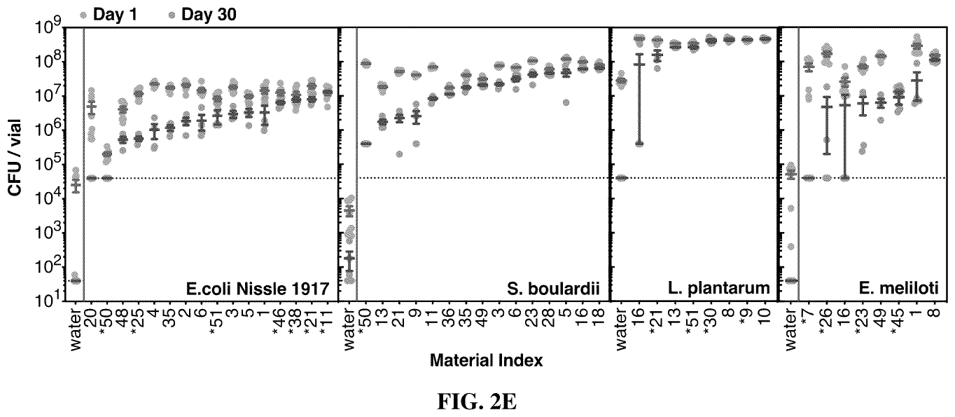

To overcome the incongruity between the commercial and academic fields, a high throughput pipeline was developed to generate synthetic extremophiles (FIG. 2A, Example 8). A synthetic extremophile was defined as a microbial formulation that had an enhanced capacity to survive insults such as drying, heating, and exposures to pressure, acid, organic solvents, and ionizing radiation. Much like natural extremophiles (e.g., spore-formers, tardigrades), such resilience would allow a synthetic extremophile to survive through the various manufacturing processes and long-term storage needs in the pharmaceutical, agricultural, and space health fields.

It was hypothesized that a synthetic extremophile that could survive in the dry state could then be enhanced to survive additional environmental insults using material formulation techniques applied to the dry powder (e.g., coating)⁹. Therefore, a library was screened, composed primarily of materials generally recognized as safe (GRAS) by the Food and Drug Administration (FDA) for their ability to protect microbes through freeze-drying and survival at room temperature for 24 hours (Table 2). This materials library was applied to 4 microorganisms (Escherichia coli Nissle 1917, Ensifer meliloti, Saccharomyces boulardii, Lactobacillus plantarum) that span a wide range of phylogenetic backgrounds, are technologically important and genetically tractable. Each microorganism was mixed with each of the 260 materials at two different concentrations to give 2,080 microbial-material formulations.

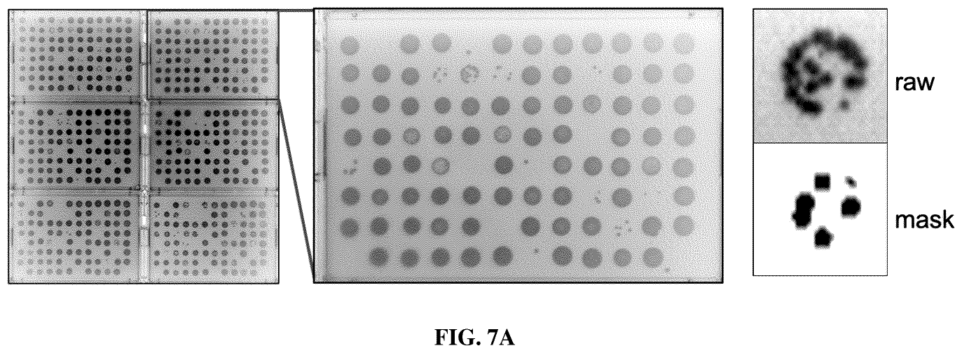

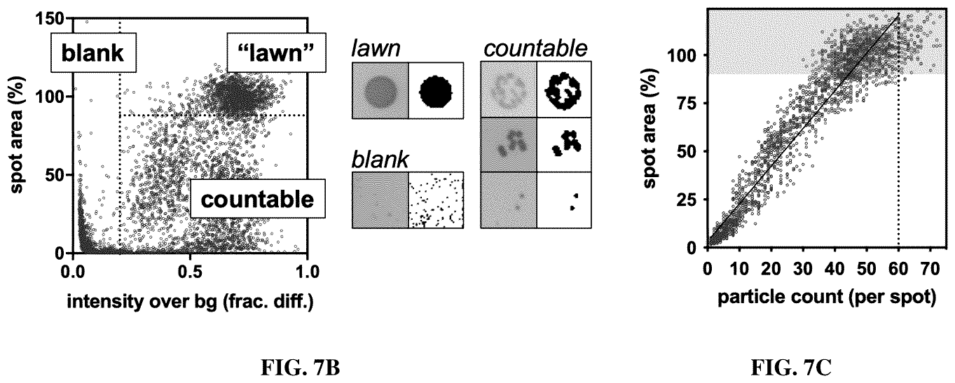

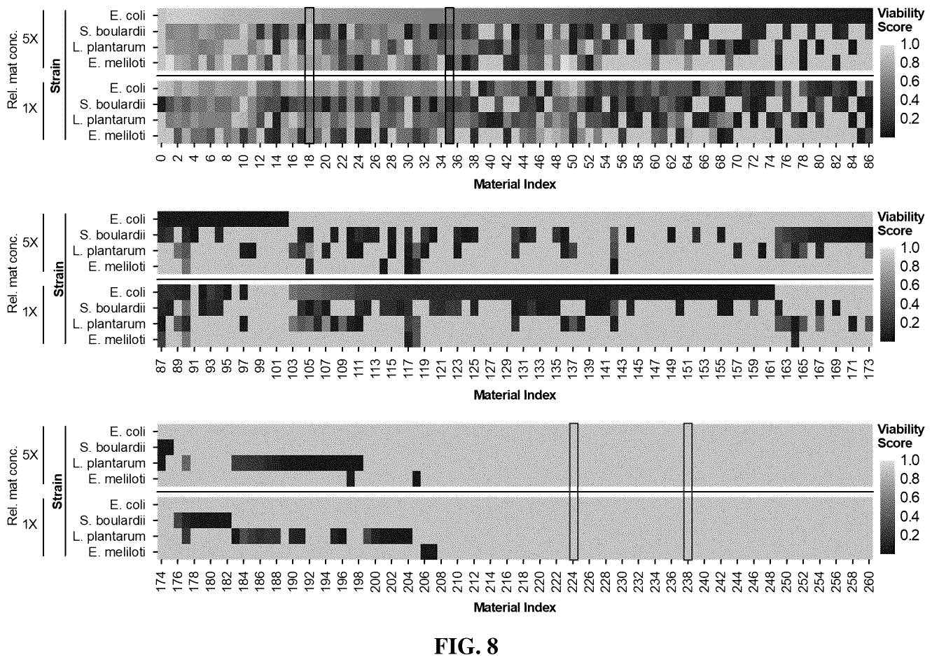

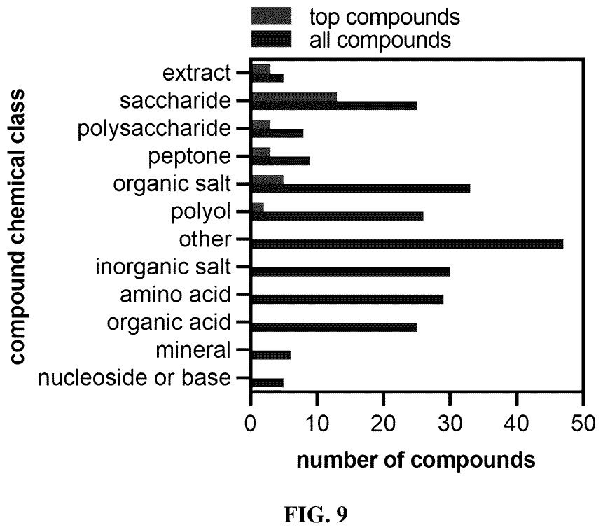

The residual viability of each formulation was measured via colony counts assessed in high throughput resulting in a normalized viability score (FIG. 2B, FIGS. 7A-7C and Example 8). The included negative controls (i.e., antimicrobial compounds sodium metabisulfite and sodium hydroxide) consistently gave viability scores of 0, and the positive controls (i.e., known lyoprotectants trehalose and ATCC reagent 20) consistently resulted in measurable viability (FIG. 2C and FIG. 8, black boxes). Additionally, sugars and peptones were overrepresented in top performing formulations (FIG. 9). Surprisingly, neither the positive controls nor any of the single materials were top performers across the range of organisms. Instead, these results show that there are ideal species-specific formulations, with low overlap in best-performing materials even between the two gram-negative representatives (i.e., E. coli Nissle 1917 and E. meliloti) (FIG. 2D).

Next, the top performing materials for each of the four microorganisms were further stratified by evaluating colony counts after extended storage for one month at room temperature and relative humidity <5% (FIG. 2E). These conditions were chosen to differentiate microbial-material combinations by their thermotolerance. This is in contrast to current microbial freeze-drying practice which still requires refrigeration to maintain acceptable viability in the dry state¹⁰. Stratifying in this way also provided a fingerprint for the intrinsic extremophilic qualities of the chosen organisms. For example, these results indicate that the lactic acid bacteria L. plantarum already had good thermotolerance as nearly all the top performing materials were indistinguishable after storage (FIG. 2E). Conversely, the two gram-negative bacteria showed a steeper drop-off with only a handful of materials maintaining the initial viability long-term. These results further corroborate the probiotic viability survey described herein, suggesting that the lactic acid bacteria that dominate the market may be naturally more tolerant and easily stabilized by a wide range of materials.

### Example 4: Optimization of the E. Coli Nissle 1917 Synthetic Extremophile

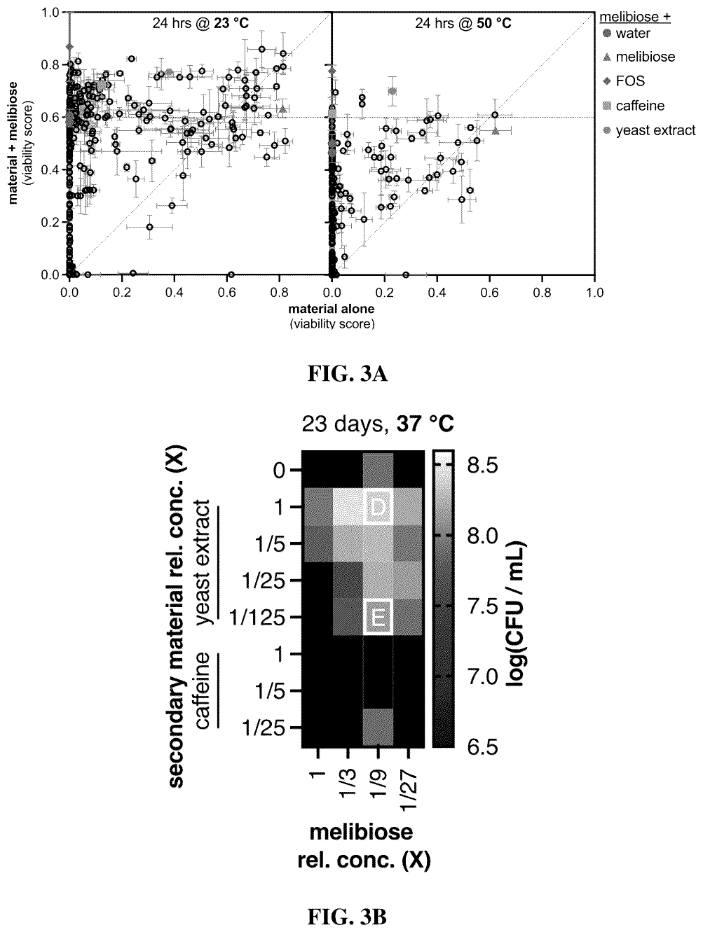

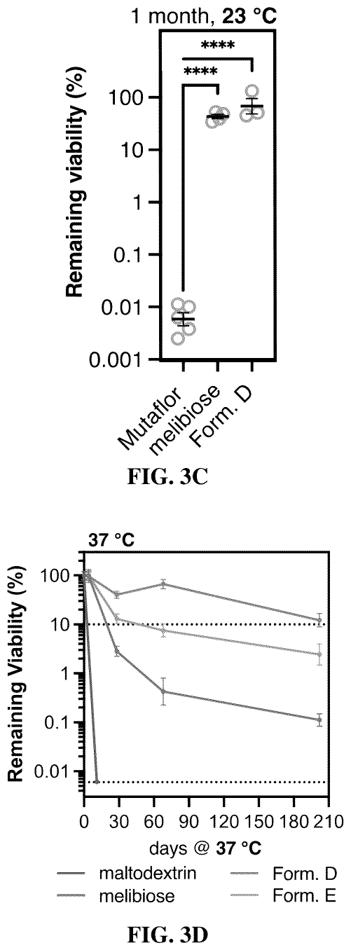

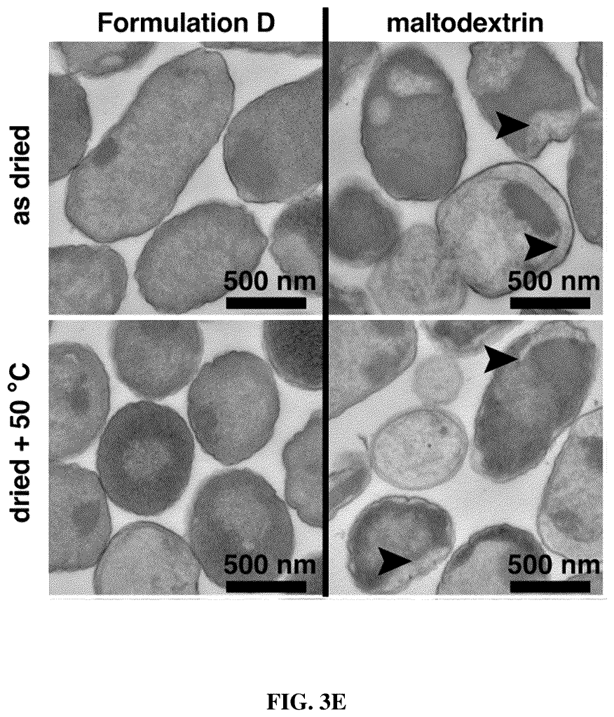

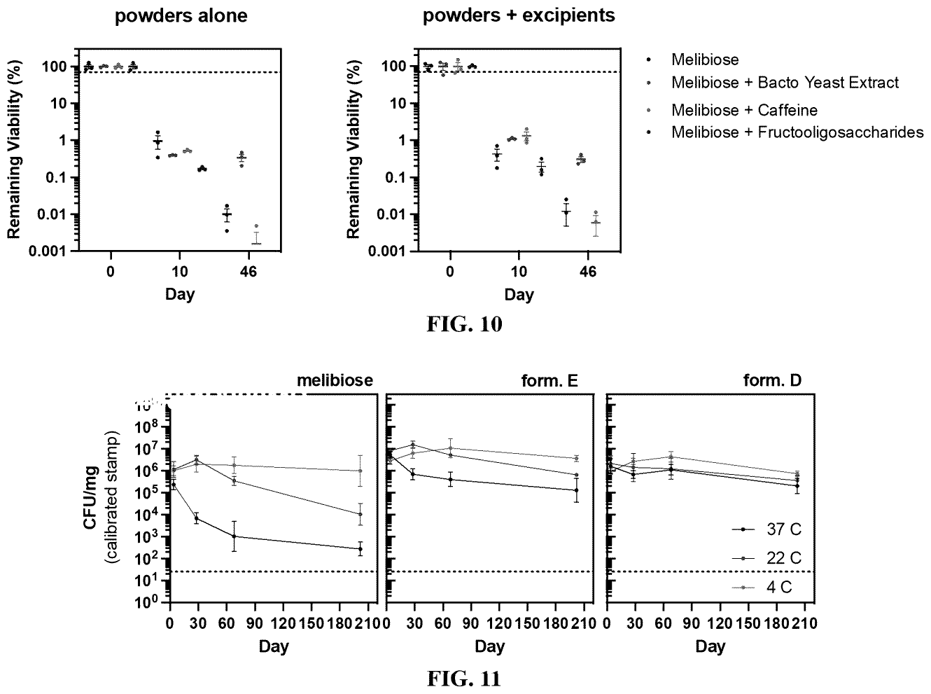

Due to its importance in the microbial therapeutics and synthetic biology fields, the extremophilic qualities of E. coli Nissle 1917 were further enhanced. This screen showed that previously established mixtures, such as the positive control ATCC reagent 18, tended to outperform the individual constituent components of the mixtures (i.e., tryptic soy broth, sucrose, and albumin for ATCC reagent 18). Therefore, two-material combinations of the library members with the best performing material (melibiose) were next compared against the single-material formulations, and the residual viability after 24 hours was evaluated both at room temperature as well as at 50° C. (FIG. 3A). Top hits were defined as material combinations with viability scores above the control (melibiose+vehicle). Library members that were themselves mixtures (e.g., LB broth) or of animal origin (e.g., mucin) were excluded to ease future manufacturing. The stability of the chosen two-material formulation (melibiose in combination with fructooligosaccharides, caffeine, or yeast extract) was analyzed in a larger format at 37° C. for extended storage times, and the mixtures with caffeine or yeast extract were found to be robust (FIG. 10). The relative concentrations of melibiose, caffeine, and yeast extract were titrated to find the optimal formulations D and E (FIG. 2B).

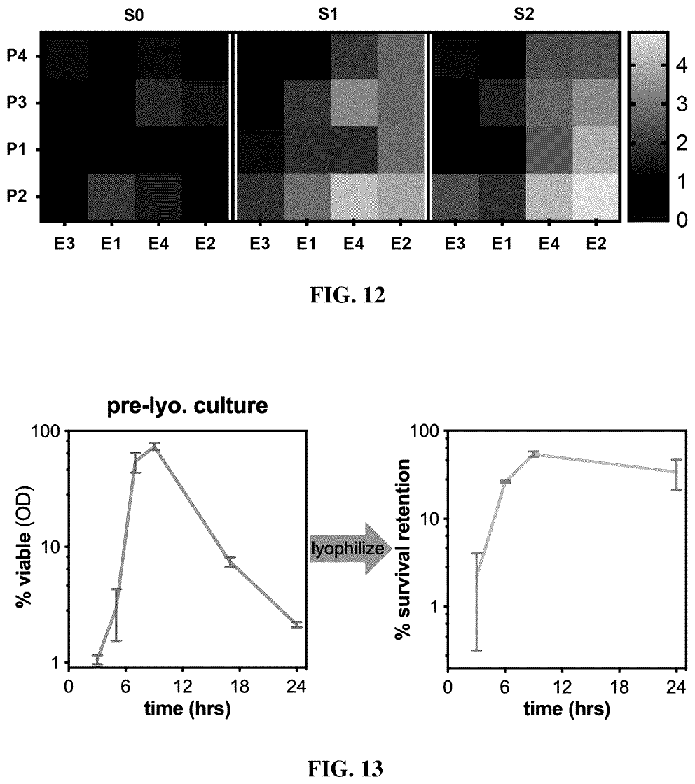

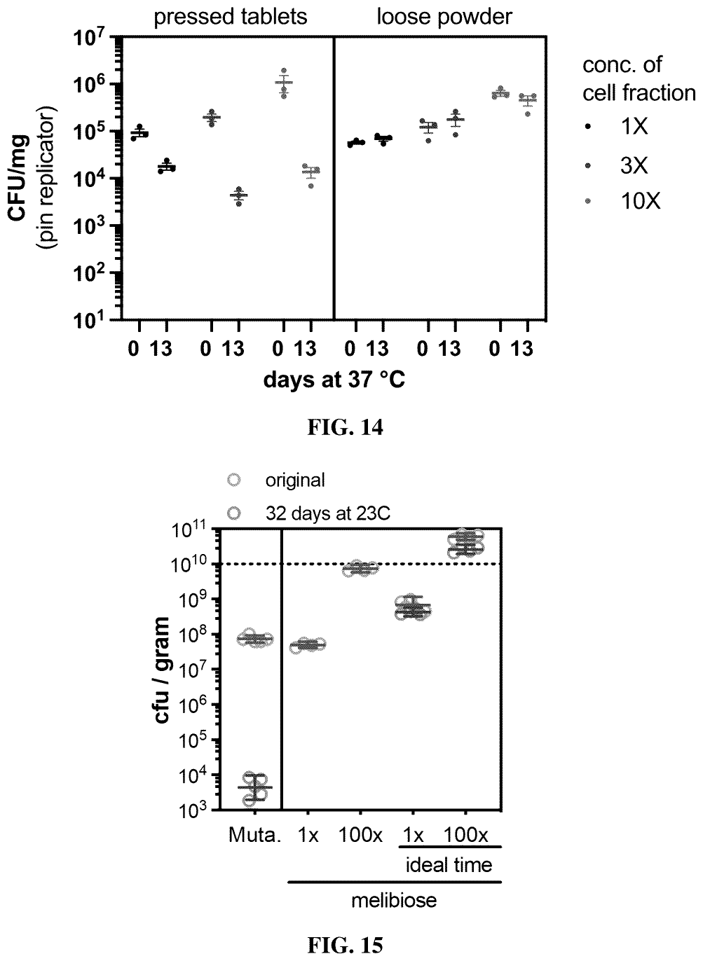

Both melibiose and formulation D outperformed the commercial product Mutaflor (E. coli Nissle 1917) by over 3.5 orders of magnitude when stored at room temperature for 1 month (FIG. 2C). To confirm these results and exclude any manufacturing bias (i.e., freshly made vs store-bought product), E. coli Nissle 1917 was formulated in maltodextrin (the stabilizer used in Mutaflor) using the presently disclosed freeze-drying processes. This “commercial-like” dry E. coli Nissle 1917 was compared with the top performing formulations. It was found that at 37° C. the maltodextrin stabilized material lost all measurable viability (>4 orders of magnitude) within 11 days (FIG. 2D). In contrast, formulation D retained over 10% viability even after 6.5 months at 37° C. (FIG. 2D and FIG. 11). This was >2 orders of magnitude better than the unoptimized melibiose formulation. In addition to maintenance of relative viability it was also shown that the absolute CFU count could be optimized by altering the growth medium (FIG. 12), time of harvest (FIG. 13) and percent loading of bacteria (FIG. 14) to produce synthetic extremophiles with viabilities >10¹⁰ CFU per gram (FIG. 15).

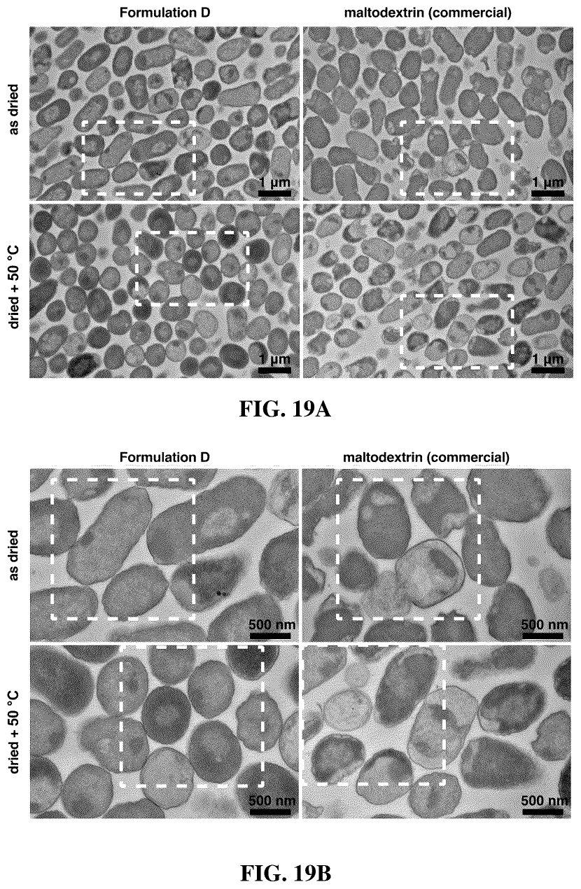

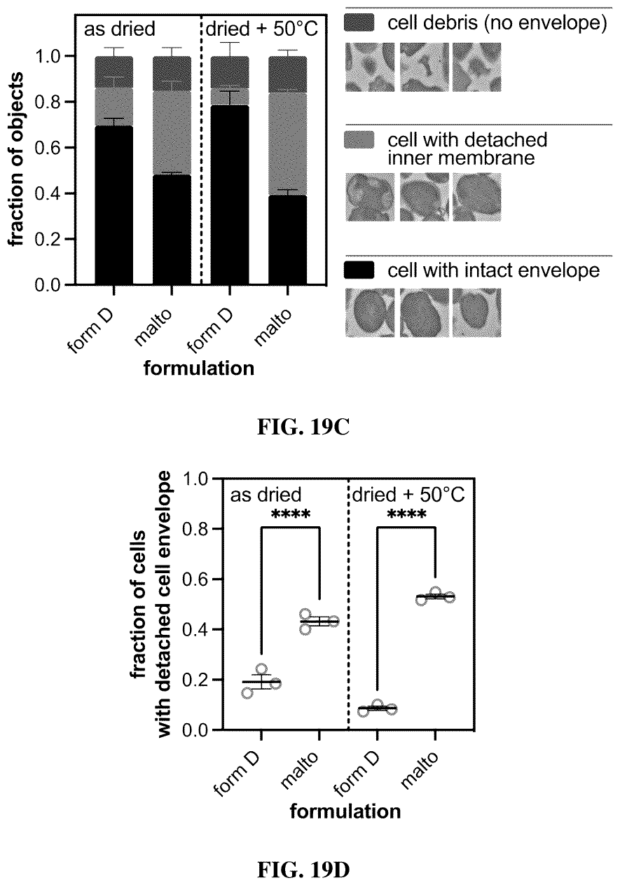

To understand the mechanism of protection, transmission electron microscopy (TEM) was used to compare the ultrastructure of E. coli Nissle 1917 cells dried in either the well-performing formulation D or the poor-performing commercial formulation (maltodextrin). The formulation D samples had a significantly lower fraction of cells with cell envelope defects (i.e., detachment of the inner membrane from the outer membrane with the periplasmic space filled with electron-dense material) (FIGS. 3E and 19A-19D). This difference between formulation D and maltodextrin was larger for samples exposed to 50° C. (FIG. 19D). While protection against this membrane defect may arise via several mechanisms, one potential mechanism may involve stabilization of membrane proteins critical for survival of the rehydration process. This hypothesis is supported by the observation that stationary phase pssA null E. coli mutants, which lack phosphatidylethanolamine leading to misfolded membrane proteins, also develop this specific membrane defect^{23,24}.

### Example 5: Compatibility of Synthetic Extremophiles with Pharmaceutical Manufacturing Methods

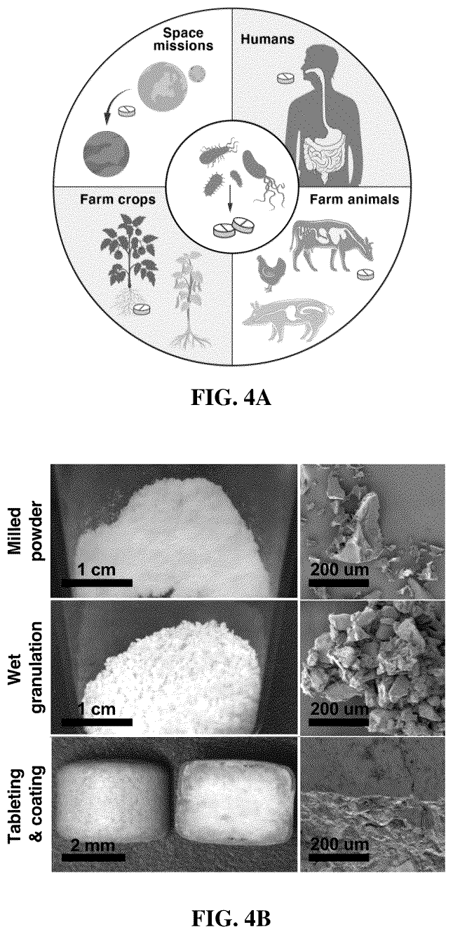

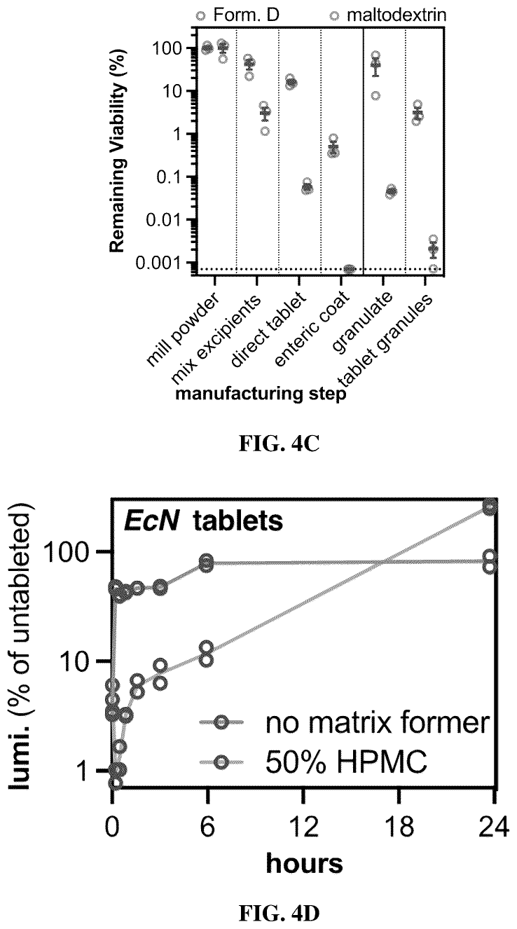

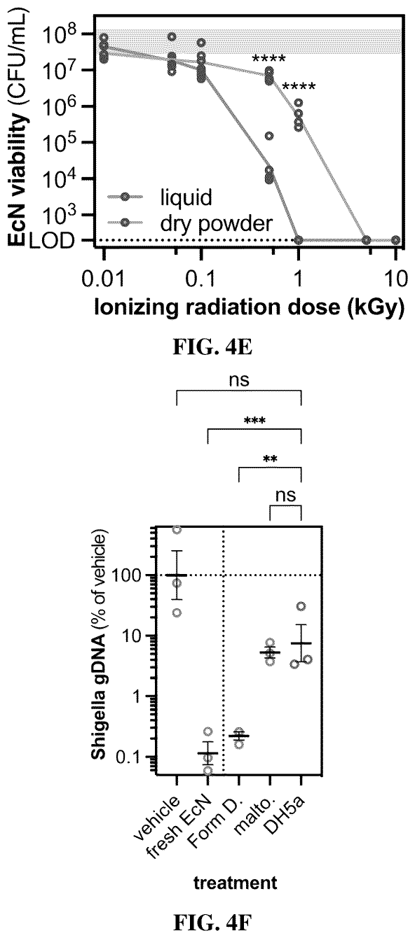

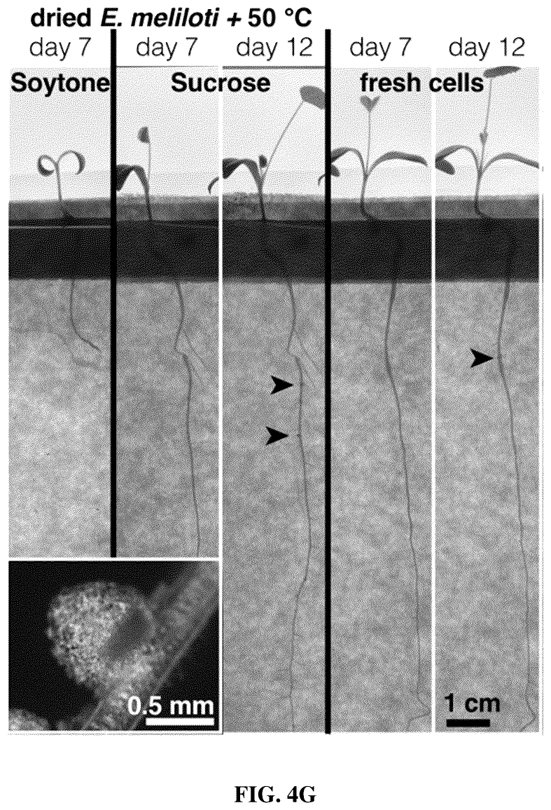

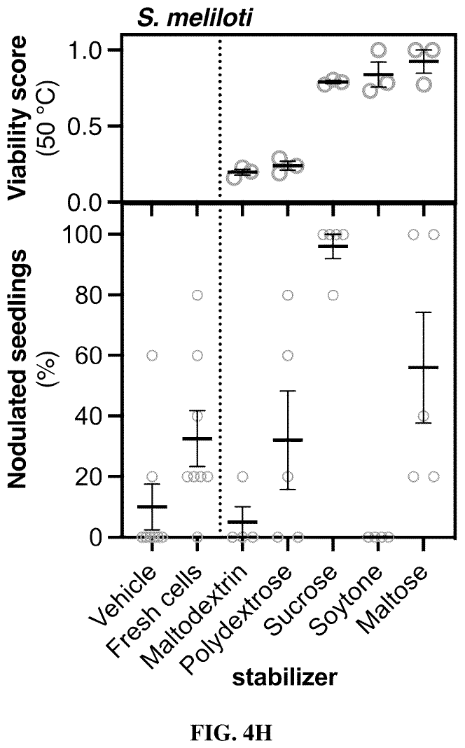

These synthetic extremophiles open the field of potential use cases for microbial products to treat disease, enhance agricultural output and support exploration space travel (FIG. 4A). One opportunity for human and animal health would be the ability make dosage forms with tuned release parameters (e.g., delayed release, enteric coatings, etc.). However, this requires applying pharmaceutical manufacturing techniques normally too harsh for living cells such as milling (exposure to shear forces), wet granulation (exposure to isopropanol and baking), tableting (exposure to high pressure) and spray coating (exposure to acetone and baking). Therefore, the E. coli Nissle 1917 synthetic extremophile was submitted to this battery of processes (FIG. 4B) and its robustness compared to the current commercial alternative (maltodextrin formulation) was characterized (FIG. 4C). It was found that the synthetic extremophile could be milled, mixed with excipients (i.e., binder, glidant, filler) and tableted in open air at room temperature and still retain >15% viability which was 2.4 orders of magnitude higher than the commercial control (0.06% viability) (FIG. 4C). Wet granulation was similarly well tolerated (>28% viability) and it was found that tableting pressure modulates the ultimate shelf stability (FIG. 15).

Once tableted, the synthetic extremophile also survived spray coating with an acetone solution of an enteric polymer (Eudragit L100-55) whereas the commercial alternative lost all viability (FIG. 2C). In this state not previously accessible by E. coli Nissle 1917, it was found that the synthetic extremophile could retain 100% viability through an exposure to simulated gastric fluid at pH 1.2 for 1 hour at 37° C. (FIG. 17).

### Example 6: Compatibility of Synthetic Extremophiles with Ionizing Radiation

Next, to validate the compatibility of the synthetic extremophile with use in exploration space flight, its hardiness was characterized by exposure to a range of ionizing radiation doses from 100 to 10,000 Gy (Typical radiation dose values: ˜15 uGy/day on Earth¹¹, ˜200 uGy/day on the Moon¹², ˜250 uGy/day on Mars¹³, total accumulated dose 500 day trip to Mars is 0.32 Gy¹⁴, 444 days outside the ISS is 0.41 Gy¹⁵). The maximum value of 10 kGy represents the dose generally accepted for radiation-based sterilization of food.¹⁶ The synthetic extremophile could sustain exposures up to 1000 Gy (FIG. 4E). At this radiation level a liquid suspension of the same bacteria lost all measurable viability.

### Example 7: Functional Characterization of Synthetic Extremophiles

Next, it was determined whether the synthetic extremophile could also remain functional in the tableted state and whether that function could be tuned through dosage form design. To test this, a bioluminescent biosynthetic pathway (luxCDABE) that produces luminescence upon rehydration was incorporated. While the dry tablet showed no measurable luminescence, 48% of the ultimate luminescent level was reached within 15 minutes of rehydration (FIG. 4D). Substituting the tablet filler by the matrix forming material hydroxypropyl methylcellulose (HPMC) led to a slow release of the bacteria and tuning of the kinetics of the luminescent function over a period of 24 hours (FIG. 4D). Combinations of immediate and slow-release tablet layers can be used to make a mixed microbial pill that seeds different bacteria in the host microbiome at different rates.

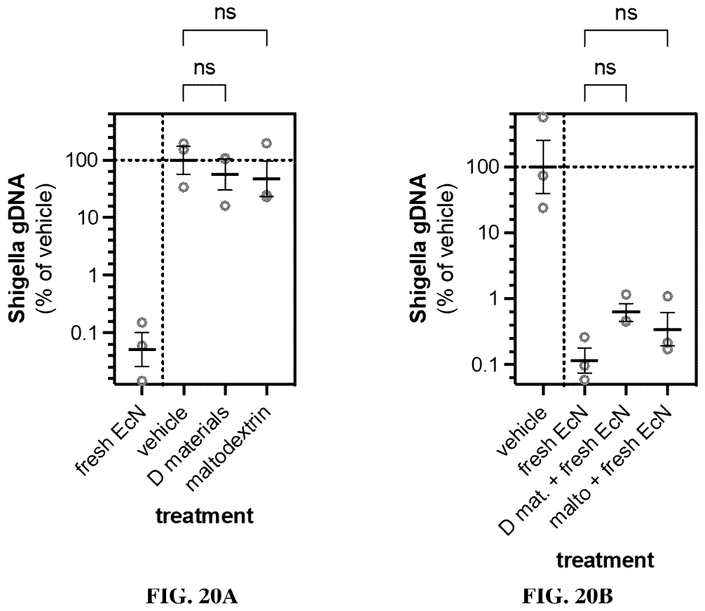

Next, it was determined whether the capacity of E. coli Nissle 1917 to inhibit enteric pathogens was impacted by the formulation²⁵. Specifically, inhibition of Shigella flexneri, the leading cause of diarrhea-associated deaths in low- and middle-income countries²⁶, was characterized. Both E. coli Nissle 1917 and its synthetic extremophile version (formulation D) inhibit the growth of S. flexneri by >99.8% in co-culture, while formulation in the commercial comparator (maltodextrin) showed no increase in effect compared to a non-inhibitory strain of E. coli (FIG. 4F). Control experiments showed that stabilizers alone did not cause inhibition, nor did they modify the inhibitory effect when added to fresh E. coli Nissle 1917 cells (FIGS. 20A-20B).

Finally, due to its potential for increasing the sustainability of agricultural systems with a lower need for chemical fertilizers, it was determined whether the presently disclosed approach to making the synthetic extremophiles would interfere with the complex biological process of symbiotic nitrogen fixation in plant roots^{3,17}. This symbiotic process occurs between legumes and bacteria (rhizobia) in the soil via the formation of root nodules which house the bacteria that can then fix atmospheric nitrogen (N₂) into a usable form for the plant¹⁷. Specifically, nodule formation by the nitrogen-fixing bacteria E. meliloti was evaluated in Medicago truncatula, a model legume used for studying this process^{17,27}.

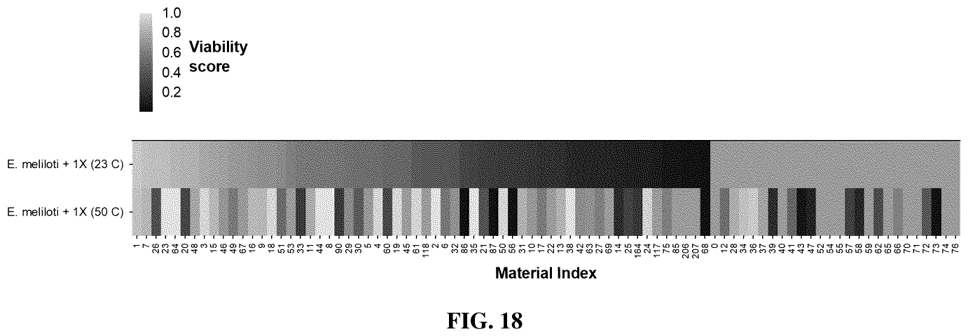

E. meliloti is currently used as a seed inoculant prior to planting, however, there remains a need for improved stability of the microbial product present during formulation, storage, and seed inoculation, particularly when it comes to tolerance to elevated temperatures above 40° C.^{28,29}. Therefore, to generate a synthetic extremophile version of E. meliloti, the heat shock screen was repeated at 50° C. to find materials that would give E. meliloti synthetic thermotolerance (FIG. 18). From these, three optimal materials (sucrose, soytone, maltose) and two suboptimal materials (maltodextrin, polydextrose) were chosen. E. meliloti was formulated in each, submitted to a 50° C. shock for 24 hours, rehydrated, and applied to seedlings of M. truncatula A17 (FIG. 4G). Functional symbiosis was measured as percent of seedlings that successfully formed nodules. The synthetic E. meliloti extremophiles made with sucrose and maltose were able to successfully nodulate, while the extremophile made from soytone lead to plant stunting (FIGS. 4G-4H). These results show that the high throughput pipeline described herein can define a set of materials that are not just ideal for the target microbial species, but which can be further selected for those that are compatible with the specific target application that may include complex biological processes such as nodulation.

In sum, presented herein is an approach for the identification of materials capable of imparting microorganisms with extreme environmental tolerance. These synthetic extremophiles stand to transform the capacity to disseminate bioactive organisms across human applications, from shelves across the globe, to fields for agricultural practices to shuttles for space exploration.

### Example 8

Viability Survey of Commercial Probiotic and Microbial Products (FIG. 1, FIGS. 5a-5b).

All products were purchased and analyzed before their expiration dates. The specific product names, lot numbers, expiration dates, date of analysis and dosage forms are summarized in Table 1. All procedures were analyzed under sterile conditions. The dosage forms encountered were two-piece capsules filled with loose powder, sachets (packets) filled with loose powder or tablets. In each case only the microbial fraction was analyzed. Specifically, for capsules, the two halves were carefully separated, and the internal contents poured out onto weighing paper for further analysis. For sachets, the sachets were carefully torn open, and the internal contents poured out onto weighing paper for further analysis. For tablets, the tablet was placed in a sterile zip-loc bag, crushed by manually rolling a plastic tube over the tablet, and the resulting powder was poured out onto weighing paper for further analysis.

Promised colony forming unit (CFU) counts per dose were recorded from the product packaging and converted to CFU per gram by dividing by the mean mass of 4 doses. For Florastor, the amount of yeast cells was reported in milligrams per dose, and this number was converted to CFU per dose by dividing by the approximate cell dry weight of yeast (˜20 pg, 32% of 60 pg)[BNID 101795, BNID 105094]¹⁸. The dose mass only includes the recovered microbial fraction as described above (i.e. internal contents of the dosage form). In cases that products report a CFU count both “at manufacture” and “by expiration date” only the CFU count “by expiration date” was used. This data is summarized in Table 1.

Viable CFU counts per dose were determined by rehydrating one dose in phosphate buffered saline (PBS, ThermoFisher 10010049) at the specified rehydration ratio on ice, making 10-fold serial dilutions in PBS on ice, plating 100 μL of each dilution onto the appropriate solid medium in 100 mm circle petri dishes with 4.5 mm plating beads (Zymo), incubating at the appropriate conditions, and counting colonies on the dilution that gave ˜200-1000 distinct colonies. Four or five separate doses of each product were prepared as described. As noted in Table 1, the rehydration ratio was typically one dose in 50 mL, but in a few instances the rehydration volume was lowered when the cell counts were found to be low during an initial analysis. The medium and culture conditions used for each product were chosen based on the microorganisms listed on the package and are listed in Table 1. Medium components were all purchased from BD Life Sciences as specified in Table 3, solidified with 1.5% w/v Bacto Agar (BD, 214010) and prepared according to the label instructions. For aerobic conditions plates were incubated in a static incubator and for anaerobic conditions plates were first placed in a BD GasPak EZ Gas Generating Systems container (BD, 260002) with an anaerobe sachet (BD, 260001) and incubated in a static incubator. Colony counts were determined systematically by first imaging the plates in a gel imager (ChemiDoc XRS+, BioRad) using UV transillumination and the standard emission filter (580/120), and then programmatically counting the colonies using FIJI (imageJ)¹⁹. Total viable CFU counts per dose were calculated by multiplying the colony counts by the plated dilution factor (to get a CFU/mL value) and then the total rehydration volume. Viable CFU counts per dose were then converted to CFU counts per gram by dividing by the mass of the specific dose analyzed. A dose only includes the recovered microbial fraction as described above (i.e., internal contents of the dosage form).

Total cell counts per dose were determined using an automated microscopic cytometer (QUANTOM Tx Microbial Cell Counter, Logos Biosystems) according to the manufacturer protocols. Specifically, a 10 uL sample of the rehydrated dose used for the viable CFU counts (above) was mixed with 2 uL of a 1:1 mix of total cell staining dye and enhancer (Logos Biosystems, Q13501) on ice, then 8 uL of cell loading buffer (Logos Biosystems, Q13501) and 6 uL of the mixture were loaded onto hemocytometer slides (Logos Biosystems, Q12001). Slides were centrifuged for 10 minutes at 300 rcf in a slide microcentrifuge (Logos Biosystems, Q10002) and then loaded into the cell counter. Four or five separate doses of each product were prepared as described, and each prepared sample was loaded and quantified in duplicate. For products that gave counts above 1e9 cells per mL during an initial analysis, the rehydrated dose was first diluted 10-fold in PBS before sample preparation. Total cells per dose were calculated by multiplying the cells per mL as reported by the instrument by the dilution factor (if any) and the total rehydration volume as specified in Table 1. Total cell counts per dose were converted to cell counts per gram by dividing by the mass of the dose analyzed. A dose only includes the recovered microbial fraction as described above (i.e., internal contents of the dosage form).

Percent viable cells relative to promised cells were calculated by dividing the viable CFU counts per gram by the promised cell counts per gram. Percent viable cells relative to total cells were calculated by dividing the total CFU counts per gram by the total cell counts per gram.

Heat Stress Test of Commercial Probiotic and Microbial Products (FIGS. 6a-6b)

Three or four doses of each product as listed in Table 1 were incubated at 50° C. for 24 hours in their original dosage form in a static incubator. Three or four control doses were kept at the manufacturer recommended storage temperature (4° C. or 23° C. as noted in FIGS. 6A-6B) for the same period. The viable CFU counts per gram for heat stressed and control doses were determined as described above (“Viability survey of commercial probiotic and microbial products”). Percent retention of viability was calculated by dividing each heat stressed CFU count per gram by the mean of the control CFU counts per gram.

### Microbial Strains Used for Stabilization and General Culture Conditions

Details of microbial strains used are listed in Table 4 and medium components are listed in Table 3.

E. coli Nissle 1917 was isolated from the commercial product Mutaflor and routinely cultured on LB agar at 37° C. in a static incubator or LB broth in 14 mL culture tubes or vented, baffled culture flasks shaken at 250 rpm at 37° C. in a shaker incubator.

S. boulardii was isolated from the commercial product Florastor and routinely cultured on YPD agar at 30° C. in a static incubator or YPD broth in 14 mL culture tubes or vented, baffled culture flasks shaken at 250 rpm at 30° C. in a shaker incubator.

E. meliloti Rm1021 was purchased from the American Type Culture Collection (ATCC) and routinely cultured on TY agar at 30° C. in a static incubator or TY broth in 14 mL culture tubes or vented, baffled culture flasks shaken at 250 rpm at 30° C. in a shaker incubator.

L. plantarum NC8 was purchased from the Culture Collection University of Gothenburg (CCUG) and routinely cultured on MRS agar or MRS broth in sealed 14 mL culture tubes or sealed culture flasks at 37° C. in a static incubator.

S. flexneri was purchased from the American Type Culture Collection (ATCC) and cultured as described for E. coli Nissle 1917.

High Throughput Pipeline for Microbial Material Stabilizers (FIGS. 2a-2c, FIG. 3a, FIG. 4H, FIGS. 7a-7c, FIG. 8, FIG. 18)

Microbial strains were precultured overnight in 5 mL liquid medium at the appropriate conditions (see above). The overnight culture was diluted 1:1000 fold into three replicates of 125 mL of fresh liquid medium in a flask and cultured for 24 hours. The optical density (OD₆₀₀) of each replicate culture was recorded (typically 2-2.5 for E. coli Nissle 1917, E. meliloti, L. plantarum and 5-6 for S. boulardii) and the cells were pelleted at 3220 rcf for 15 minutes. The cell pellets were then resuspended in PBS (E. meliloti, L. plantarum, S. boulardii) or spent medium (E. coli Nissle 1917) to a final OD₆₀₀ of 2.25 and used immediately.

The material library was prepared by mixing each material with ultrapure water to the specified concentration (1×, 5×) in Table 2. The vendors and product numbers for all materials are listed in Table 2. The material solutions were arrayed in sealed deep well plates and kept at −20° C. until needed. On the day of use, the material plates were thawed at room temperature, vortexed well and centrifuged briefly.

For the two-material combinations with melibiose, the arrayed material library was first mixed 1:1 with melibiose or water as a control at the 1×concentration of all components (Table 2). This gives a 0.5×final concentration of each component in FIG. 3A relative to the one-material library results reported in FIG. 2C and FIG. 8.

To freeze-dry, each replicate microbial cell suspension (25 uL) was mixed with the arrayed materials (75 uL) in batch into flat-bottom 96-well plates using a liquid handling robot (Tecan, EVO 150). The arrayed microbial-material plates were immediately placed into a tray freeze dryer (Labconco, FreeZone Stoppering Tray Dryer) with shelfs pre-cooled to −40° C. and on custom 0.25-inch aluminum pedestals to ensure heat transfer to the plates. The samples were annealed at −20° C. and then dried at −20° C. and 0.1 mBar for 12 hours (nominal) and then at 37° C. for 3 hours (nominal). The chamber was backfilled with nitrogen, opened to atmosphere and then the plates were covered loosely with parafilm, capped, and placed in nitrogen-flushed zip-loc bags with desiccant (Drierite, granular −8 mesh). The bagged, freeze-dried plates were stored in the dark at the specified temperature (23° C. or 50° C.) for 24 hours.

The viability of the stored microbial-material combinations was determined by rehydrating in water, making 10-fold serial dilutions in PBS, plating onto the appropriate solid medium and quantifying the resulting colonies. Specifically, using a liquid handling robot (Tecan, EVO 150), each replicate plate was rehydrated with ultrapure water (200 uL per well), diluted in PBS to make 1:10², 1:10³, 1:10⁴ dilutions (relative to the initial volume before drying) and 4 μL of each of the dilutions were spotted onto solid medium in 1-well rectangular plates. After incubation at the appropriate culture conditions (see above) the plates were imaged in a gel imager (ChemiDoc XRS+, BioRad) using UV transillumination and the standard emission filter (580/120).

To assign a Viability Score, using FIJI (imageJ), the 16-bit tiff plate images were programmatically separated into individual spot images, each spot was segmented, and several image attributes were measured (raw particle count, mean background value, mean segmented region value, segmented region area). These were used to classify spot images into empty, countable and lawns using the mean segmented region value to background value ratio and segmented region area attributes (FIGS. 7A-7C). Raw particle counts for the empty class were set to 0. Raw particle counts for the countable class was interpreted as a true colony count. Raw particle counts for the lawn class were set to a microbe-specific colony count value determined by extrapolating the data observed on a raw count vs segmented region area plot (FIGS. 7A-7C). For E. coli Nissle 1917 and E. meliloti lawns were set to 60 counts. For S. boulardii lawns were set to 50 counts. For L. plantarum lawns were set to 120 counts. These values are sensitive to the colony size at the time of analysis. Finally for each microbial-material combination a Viability Score was calculated, which is the cumulative sum of colony counts at the three dilutions divided by the maximum total possible count (i.e., all lawns). The Viability score thus ranges from 0 to 1. This method allows for the use of the data across all three analyzed dilutions in order to average out the sampling noise caused from plating and counting low numbers of colonies (i.e., the three dilutions act as technical replicates). It also allows for agnosticism with respect to the actual number of countable colonies on any dilution and assignment of a robust value to microbial-material combinations that lead to lawns in some dilutions and biological replicates (i.e., can assign a value to a wider range of viabilities that might be possible by using only the countable spots). This analysis was applied to three independent biological replicates for each material-microbial combination.

Correlation analysis (FIG. 2D) of top performing materials for each organism was carried out by first normalizing the viability scores of each specific material and concentration to the maximum viability score observed for each organism. This gives a list of materials (at specific concentrations) for each organism with normalized scores that span the full range 0 to 1. Then any material with a normalized score above 0.75 was defined as a top material for that organism. Based on which organism sets each of these top performing materials (at specific concentrations) belonged to, they were binned into the 15 possible sets of 1-, 2-, 3- and 4-organism overlaps.

### Vial-Based Viability Validation of Microbial Material Stabilizers (FIG. 2E)

Top performing microbial-material combinations in the high throughput pipeline were assayed at a larger scale to determine a more accurate relative viability value (CFU/vial) and assess their short term (1 day) and long term (30 day) stabilization potential at room temperature. The analysis described below was repeated for 7-8 replicates for the 1 day time point and for 4-5 replicates for the 30 day time point.

Microbial strains were precultured overnight in 5 mL liquid medium at the appropriate conditions (see above). The overnight culture was diluted to an OD₆₀₀ of 0.00225 in 25 mL of liquid medium in a flask and cultured for 24 hours at the appropriate conditions (see above). The cells were harvested by centrifugation and resuspended in cold PBS at a final OD₆₀₀ of 2.25 and used immediately.

To freeze-dry, each microbial cell suspension (100 uL) was mixed with the target material (300 uL) with a micropipette in autoclaved threaded tube vials (Electron Microscopy Sciences, 60304-04) capped with 2-prong lyophilization stoppers (Electron Microscopy Sciences, 60304-41) to the first stop. Within 10 minutes of mixing, the vials were placed in a tray freeze dryer with shelfs pre-cooled to −40° C. and in aluminum StableTemp vial blocks (Cole-Parmer, EW-36600-44). Samples were dried as described above (see “High throughput pipeline”). After drying, the chamber was backfilled with nitrogen, opened to atmosphere, vials fully stoppered, sealed with screw caps, and stored in the dark at 23° C. for the specified time.

The viability of the stored samples was assessed by hydrating the samples with 4 mL of cold PBS (to give a 1:10 dilution relative to the original volume), making 10-fold serial dilutions, plating 100 uL of each dilution onto the appropriate solid medium in 100 mm circle petri dishes with 4.5 mm plating beads (Zymo), incubating at the appropriate conditions, and counting colonies on the dilution that gave ˜200-1000 distinct colonies. CFU counts per vial were determined systematically as described above using a gel imager and FIJI (see “Viability survey of commercial probiotic”).

### Validation of Two-Material Combinations (FIG. 10)

E. coli Nissle 1917 was prepared as described above (see “Vial-based viability validation”) with a few modifications: the flask culture was 2 L and incubated for 17-18 hours and the final cell suspension in PBS was set to an OD₆₀₀ of 10-12.

To freeze-dry, the cell suspension (12.5 mL) was mixed with the target material combination (37.5 mL), poured into rectangular 1-well plates, and placed into a tray freeze dryer with shelfs pre-cooled to −40° C. and on custom 0.25-inch aluminum pedestals to ensure heat transfer to the plates. Samples were dried as described above (see “High throughput pipeline”). After drying, the chamber was backfilled with nitrogen, opened to atmosphere, plates were transferred to zip-loc bags, the dry material was scrapped into the bag, milled by rolling a plastic tube on the outside of the bag and the resulting powder was kept in the bag with a desiccant pack and stored in the dark at 4° C. until needed.

To prepare samples “with excipients”, the dried bacterial powders were mixed with 1% w/w magnesium stearate, 5% w/w polyvinylpyrrolidone and 64% w/w lactose to give a 30% w/w loading of the bacterial material.

Long-term storage of samples was done by placing ˜20 mg (for bacterial powders alone) or ˜60 mg (for bacterial powders with excipients) aliquots in 12-well plates, capping the plates and placing them in nitrogen-flushed zip-loc bags with desiccant (Drierite, granular −8 mesh). Bagged plates were stored at 37° C. in the dark for the indicated time.

The viability of the stored samples was assessed by hydrating the samples with 1 mL PBS, making 10-fold serial dilutions, spotting ˜1.5 uL of each dilution in triplicate onto LB agar in 1-well rectangular plates using a pin replicator (V&P Scientific, VP 407A), incubating at 37° C., and counting colonies on the strongest dilution that gives >1 colony per spot. Total CFU counts per sample were calculated by first dividing spot counts by the spotted volume and multiplying by the dilution factor and total rehydration volume, and then averaging across the three spotting replicates. Total CFU counts per mass were calculated by dividing the CFU counts per sample by the mass of the stored sample. Three independently stored replicate samples were analyzed as described. The percent viability at each time point was calculated by dividing the total CFU counts per mass by the mean total CFU counts per mass of three replicate samples at day 0. This method provides a robust measure of relative viability.

### Optimization of Two-Material Formulations (FIG. 3B)

To find optimal concentrations for each of the components in the selected two-material formulations for E. coli Nissle 1917, a combinatorial library of component concentrations was screened using the high throughput pipeline described above (see “High throughput pipeline for microbial material stabilizers”) with a few modifications: the flask culture was 2 L and incubated for 17-18 hours, the final cell suspension in PBS was set to an OD₆₀₀ of 10-12, dried plates were stored at 37° C. for 23 days, viability was assessed at the 1:10⁵ dilution where only empty or countable spots were present and the raw counts were converted directly to CFU/mL by dividing the spot counts by the spotted volume (4 uL) and multiplying by the dilution factor.

The top performing combinations were named Formulation D (1/9×melibiose+1×yeast extract) and Formulation E (1/9×melibiose+1/125×yeast extract).

Direct Comparison of Synthetic Extremophile E. Coli Nissle 1917 with Mutaflor (FIG. 3C, FIG. 15)

Stabilized E. coli Nissle 1917 was prepared as described above (see “Validation of two-material combinations) with either melibiose (1×) or Formulation D (see “Optimization of two-material formulations”). For 100×cultures the 2 L culture was concentrated 100× in PBS before mixing with the stabilizers. Mutaflor capsules were kept intact as manufactured during storage.

Samples were stored in nitrogen-flushed zip-loc bags, with desiccant (Drierite, granular −8 mesh), kept at 23° C. in the dark for the indicated time.

The viability of the stored samples was determined as described above (see “Viability survey of commercial probiotic and commercial products”).

### Ultra-Long-Term Assessment of Viability (FIG. 3D, FIG. 11)

Stabilized E. coli Nissle 1917 was prepared as described above (see “Validation of two-material combinations) with either melibiose, Formulation D, Formulation E or 5% w/w maltodextrin (16.5-19.5 DE) as the commercial control. It was determined from the list of inactive ingredients that maltodextrin was the only stabilizer used in the E. coli Nissle 1917 product Mutaflor.

Multi-gram samples of the dry powders were stored in 20 mL glass with desiccant and kept in the dark at the specified temperature. Viability was assessed by retrieving a small quantity (˜60 mg) of the stored powders and following the procedure described above (see “Validation of two-material combinations”) but the samples were rehydrated in ultrapure water instead of PBS. In addition to the pin replicator spotting, one sample was also traditionally plated onto 100 mm circle petri dishes as described above (see “Validation of two-material combinations”) to determine the scaling factor between the spotting method and the petri dish plating. When CFU/mg is reported, the scaled values were used. Percent viability is not impacted by this scaling factor as it is a relative value.

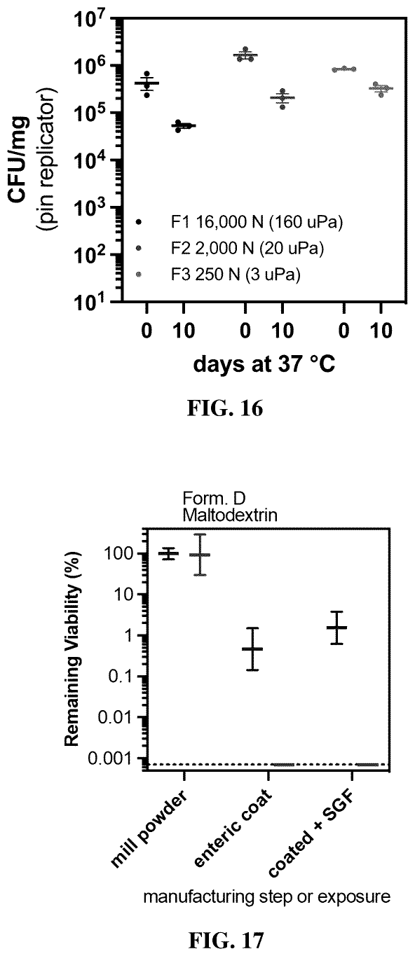

Viability after Exposure to Wet Granulation, Tableting and Enteric Coating (FIGS. 4b-4c, 16)

Stabilized E. coli Nissle 1917 was prepared as described above (see “Validation of two-material combinations) with either Formulation D or 5% w/w maltodextrin (16.5-19.5 DE) as the commercial control. Dry powders were mixed with the following excipients: 1% w/w magnesium stearate, 5% w/w polyvinylpyrrolidone and 64% w/w lactose (this gives a 30% w/w loading of the bacterial material).

Wet granulation was carried out by adding 600 uL isopropanol to 2 g of the bacterial-excipient mixture while it was being continually stirred with a stand mixer (Sunbeam, B000COC69C) room temperature. Stirring was allowed to continue for an additional 5 minutes, and the resulting paste was passed through a bench top oscillating granulator (ERWEKA, FGS II) with a 1 mm mesh screen. The collected granules were dried at 45° C. for 20 min in a food dehydrator (Magic Mill, MFD-9100) retrofit with a HEPA filter (Vornado, MD1-0022) to maintain sterility. Granules were stored in tubes with desiccant at 4° C. until used.

Tableting was carried out in the open air on a tablet press (Natoli, NP-RD10A) with a 5×5 mm circular punch and die, set to 8 mm depth, and pressed with 17 kN of force (3.4 kN per tablet equal to a pressure of 173 uPa or the indicated pressure). Either the bacterial-excipient mixture was tableted directly (“direct tablet”) or the granulated mixture was tableted (“tableted granules”). Tablets were stored in tubes with desiccant at 4° C. until used.

Enteric coating of the tablets was done by spray coating with a solution of 3.6% w/w of Eudragit S100 (Evonik) in a 1:1 co-solvent of acetone and isopropanol with 0.36% w/w triethyl citrate as a plasticizer. Spray coating was carried at 23° C. via a spray nozzle with a 0.5 mm opening with the tablets in a rotating pan coater (ERWEKA, DKE) over the course of 1 hour. Coated tablets were dried at 40° C. for 2 hours in a food dehydrator as described above. Coated tablets were stored in tubes with desiccant at 4° C. until used.

The viability of each of the processed samples was assessed as described above (see “Validation of two-material combinations”) but samples were rehydrated in water instead of PBS, and the CFU per mass was calculated by dividing by the mass of only the bacterial fraction (i.e., 30% of total mass for samples that include excipients). Coated tablets were first cut in half to expose the center before rehydration. Three independent samples of each type were assessed as described.

Viability after Exposure to Simulated Gastric Fluid (FIG. 17)

Enteric coated tablets of E. coli Nissle 1917 were prepared as described above (see “Enteric coating”) were placed in 40 mL of simulated gastric fluid (USP, pH 1.2, no enzymes) in a 50 mL conical bottom tubes and incubated for 1 hour at 37° C. on an oscillating tube revolver (Thermo Scientific, 88881001) inside a static incubator. After exposure to simulated gastric fluid, tablets were blotted dry, cut in half and the viability was assessed as described above (see “Validation of two-material combinations”).

Viability after Exposure to Ionizing Radiation (FIG. 4E).

Stabilized E. coli Nissle 1917 was prepared as described above (see “High throughput pipeline for microbial material stabilizers”) with a few modifications: only melibiose (lx) was used as the stabilizer, 32 replicate samples were arrayed on eight 96-well plates (4 replicates per plate) and bagged plates were stored at 4° C. until used. On the day of irradiation, a fresh overnight 5 mL culture of E. coli Nissle 1917 was arrayed onto the same plates (4 replicates per plate) as a control.

The plates were sequentially placed into a Gammacell irradiator (Best Theratronics, 40 Exactor) and exposed to a Cobalt-60 source for the time required to achieve the specified ionizing radiation dose (dose rate=37.12 Gy/min). One plate was maintained with the other plates but not exposed to any radiation.

The viability of each of the samples was assessed as described above (see “Validation of two-material combinations”) except that samples were rehydrated with 200 uL of PBS directly in the treatment plates to give an initial 1:2 dilution for subsequent serial dilution.

### Tuning of Release Kinetics Via Matrix Formers (FIG. 4D)

E. coli Nissle 1917 was transformed with a plasmid encoding a constitutively expressed pathway for bioluminescence (luxCDABE) via electroporation. Plasmid and strain details are listed in Table 4.

Luminescent E. coli Nissle 1917 stabilized with Formulation D was prepared as described above (see “Validation of two-material combinations) with cultures supplemented with 100 ug/mL of ampicillin. The resulting bacterial powder was mixed either with the standard excipients (1% w/w magnesium stearate, 5% w/w polyvinylpyrrolidone and 64% w/w lactose) or with the inclusion of a matrix former (50% w/w hydroxypropyl methylcellulose, 1% w/w magnesium stearate, 5% w/w polyvinylpyrrolidone and 14% w/w lactose). In both cases the bacterial material maintains a 30% w/w loading.

A fraction of each of these two powder mixtures was made into tablets as described above for the “direct tablet” method. Then either one tablet or the equivalent mass of loose powder (with excipients) was placed into 30 mL of ultrapure water in a conical bottom tube and incubated for 24 hours at 37° C. on an oscillating tube revolver (Thermo Scientific, 88881001) inside a static incubator. At the specified timepoints 200 uL samples were taken, arrayed in white-walled, clear-bottom 96-well plates and the luminescence quantified on a luminometer (Tecan, Infinite 200). Each of the four sample types (standard or matrix forming excipients in loose or tableted form) were analyzed as described in duplicate.

To calculate the percent released luminescence, at each time point the luminescence value of the tableted samples was divided by the mean luminescence value of the corresponding loose powder samples.

Shigella flexneri Inhibition Assay (FIG. 4F)

Dry stabilized E. coli Nissle 1917 was prepared as described above (see “Validation of two-material combinations) with either Formulation D or 5% w/w maltodextrin (16.5-19.5 DE) as the commercial control. Control “material-only” samples were prepared in the same way but omitting the bacterial cells. Dry powders were kept at 4° C. until use. Fresh S. flexneri, E. coli Nissle 1917 and E. coli DH5a were prepared by culturing overnight in LB at 37° C., pelleting and resuspending in fresh LB at an OD₆₀₀ of 3.

On the day of the experiment, the dry powders were rehydrated in 500 μL of deionized water (20 mg/mL), diluted to 1.5 mL total volume in water, and mixed with 1.5 mL of 2×LB. For the fresh cell samples (E. coli Nissle 1917 and DH5a), 3 uL of the concentrated overnight culture was mixed with 1.5 mL water and then with 1.5 mL of 2×LB to match the rehydrated dry samples. The vehicle control was prepared by mixing 1.5 mL of water with 1.5 mL of 2×LB.

These cultures were incubated in triplicate at 37° C. for 2 hours, and then challenged with 3 uL of the concentrated overnight culture of S. flexneri and co-cultured for 10 hours at 37° C. To quantify the remaining viable fraction of S. flexneri, the co-cultures were diluted 1:100 into fresh LB (3 mL) outgrown for 12 hours at 37° C., and total DNA extracted (Promega Wizard DNA purification kit). Shigella DNA was quantified by qPCR on a Roche LightCycler 96 using the FastStart Essential DNA Green Master Mix (Roche, 06924204001) and the following primers: forward -CCTTTTCCGCGTTCCTTGA, reverse -CGGAATCCGGAGGTATTGC.

The resulting Ct values were converted to DNA concentrations using a standard curve of purified S. flexneri DNA. For each experiment the DNA concentrations were normalized to the mean of vehicle control replicates, setting it to 100%.

Plant Nodulation Assay with Stabilized E. Meliloti (FIGS. 4g-4h)

E. meliloti cultures were prepared as described above (see “High throughput pipeline for microbial material stabilizers”) and freeze-dried with the specified stabilizers following the 1-well plate method described in “Validation of two-material combinations”. The bagged, stabilized powders were exposed to 50° C. for 24 hours in a static incubator and then kept in the dark at 4° C. until used.

Nodulation of Medicago truncatula A17 was assayed according to a protocol from Sadowsky et al. modified to growth in pouches²⁰⁻²². Specifically, M. truncatula A17 seeds (Noble Research Institute) were scarified by submerging in concentrated sulfuric acid for 8-9 minutes, washed 8× with sterile water, sterilized by submerging in 8.25% sodium hypochlorite for 90 seconds, washed 8× with sterile water, allowed to imbibe in sterile water for ˜1 hour, placed on 1% agar plates, sealed with parafilm and foil, vernalized at 4° C. in the dark for 5 days and germinated at 23° C. in the dark for 20 hours. Germinated seeds with root lengths of ˜1.5 cm were transferred to sterilized growth pouches (Mega International, CYG) pre-wetted with 5 mL of 1/2×BNM medium (see Table 3). Five seeds were placed in each pouch. Pouches were placed between 1 inch foam blocks in a domed seed propagator (EarlyGrow, B07HHR5DGN) and incubated in plant growth chamber (Fisher Scientific, PR505755L) set to a day/night cycle of 25° C. for 16 hours and 21° C. for 8 hours. The chamber was kept humid with a large tray of water.

Two days after the seeds were transferred to the pouches, the resulting seedlings were each inoculated with 1 mL of the bacterial samples in 1/2×BMN. Specifically, the heat-stressed stabilized E. meliloti powders were rehydrated in 1/2×BNN at a common concentration equivalent to 10⁸ CFU/mL of the initial bacterial culture before freeze drying. Similarly, freshly grown E. meliloti cells were pelleted and resuspended in 1/2×BNM to a concentration of 10⁷ CFU/mL.

The pouches were left undisturbed for 6 days, watered with 5 mL of 1/2×BNM as necessary and then inspected for nodules every other day. On day 12, the number of nodulated seedlings in each pouch was counted. The percent nodulated seedlings was calculated by dividing the number of nodulated seedlings by the total number of seedlings in the pouch.

Transmission Electron Microscopy of Dry Stabilized Microbial Materials (FIGS. 3e, 19a-19d)

Stabilized E. coli Nissle 1917 was prepared as described above (see “Validation of two-material combinations) with either Formulation D or 5% w/w maltodextrin (16.5-19.5 DE) as the commercial control. Dry powders were kept at 4° C. (“as dried”) or incubated at 50° C. for 6.5 hours in sealed bags and then kept at 4° C. (“dried+50° C.”).

To fix the cells, 10 mg of each sample was rehydrated in 500 μL of deionized water, pelleted for 3.5 minutes at 21 k rcf, the bulk supernatant was removed leaving behind ˜20 uL, and the resulting pellet was resuspended in 30 uL of fixative (2.5% w/v paraformaldehyde, 5% w/v glutaraldehyde, 0.06% picric acid in 0.2 M cacodylate buffer). This cell suspension was pelleted for 10 min at 21 k rcf at room temperature and then transferred to 4° C. to fix overnight.

To prepare the samples for imaging, the fixed cells were washed in 0.1M cacodylate buffer, postfixed with 1% Osmium tetroxide (OsO4)/1.5% Potassium ferrocyanide(KFeCN6) for 1 hour, washed 2× in water, 1×Maleate buffer (MB) 1× and incubated in 1% uranyl acetate in MB for 1 hour followed by 2 washes in water and subsequent dehydration in grades of alcohol (10 min each; 50%, 70%, 90%, 2×100%). The samples were then put in propylene oxide for 1 hour and infiltrated overnight in a 1:1 mixture of propylene oxide and Spurr's resin (EMS). The following day the samples were embedded in Spurr's resin and polymerized at 60 degrees ° C. for 48 hours.

To image the samples, ultrathin sections (about 80 nm) were cut on a Reichert Ultracut-S microtome, picked up on to copper grids, stained with lead citrate and examined in a JEOL 1200EX Transmission electron microscope. Images were recorded with an AMT 2 k CCD camera.

To quantify the observed cell classes, three image fields from each sample type containing ˜65-95 cells were used. Cells were manually categorized into one of three classes: “cell debris (no envelope)”, “detached inner membrane” and “intact envelope” (representative examples of each are shown in FIG. 19C). Totals were quantified using the cell counter plugin in FIJI (ImageJ).

### Scanning Electron Microscopy of Dry Stabilized Microbial Materials (FIG. 4B)

The various dry microbial materials were imaged in high vacuum mode using the secondary electron detector from the Hitachi FlexSEM TM-1000 II (Tokyo, Japan). Low voltage imaging (3 kV) was used to prevent damage from electron bombardment while providing high surface detail. The powders were mounted using double sided carbon tape (Ted Pella Inc). Using JEOL USA's Smart Coater (Peabody, MA USA), gold was coated for approximately 2.5 minutes (˜4 nm of gold deposited). This conductive coating prevented excessive surface charging artifacts in images. Typical imaging conditions included a spot intensity of 50 (based on the instrument's unitless scale from 1-100) and a working distance of less than 7 mm.

### Data Analysis

Numerical data were analyzed and plotted using Prism 9 (GraphPad). Statistical tests, replicate numbers and errors are indicated for each relevant figure panel.

TABLE 1
Commercial probiotic and microbial materials analyzed

| | | | | | | Culture |
|---|---|---|---|---|---|---|
| | | | | | | conditions |
| | | Exp. | Days | Mass | Hyd. vol. | (medium, |
| Product Name | Lot # | date | until exp. | per dose | per dose | temp, O₂) |

Details for FIG. 1, FIGs. 5A-5B

| | | | | | | | | |
|---|---|---|---|---|---|---|---|---|
| Mutaflor | 830210 | Aug. 15, 2019 | 161 | 0.18 | g | 50 | mL | LB, 37° C., |
| | | | | | | | | aerobic |
| Nitragin Gold Alfalfa and | NGA19123 | Jan. 31, 2022 | 903 | 0.05 | g | 4 | mL | TY, 30° C., |
| Sweet Clover | | | | | | | | aerobic |
| Florastor | 2033 | May 1, 2021 | 816 | 0.28 | g | 50 | mL | YPD, 30° C., |
| | | | | | | | | aerobic |
| Schiff Digestive Advantage | 4345700 | Apr. 1, 2020 | 266 | 0.31 | g | 10 | mL | Nutrient, 37° C., |
| Prebiotic Fiber Plus | | | | | | | | aerobic |
| Probiotic Tablets | | | | | | | | |
| Schiff Digestive Advantage | 4167300 | May 1, 2020 | 305 | 0.54 | g | 50 | mL | Nutrient, 37° C., |
| Daily Probiotic Capsules | | | | | | | | aerobic |
| Align Probiotic Chewables | 83121453A2 | Aug. 1, 2020 | 396 | 0.19 | g | 25 | mL | MRS + cys, 37° C., |
| for Adults Supplement | | | | | | | | anaerobic |
| Align Probiotic Supplement | 83471453A1 | Sep. 1, 2020 | 448 | 0.19 | g | 25 | mL | MRS + cys, 37° C., |
| 24/7 Digestive Support | | | | | | | | anaerobic |
| Phillips' Colon Health | 8H17A | Aug. 1, 2020 | 400 | 0.36 | g | 25 | mL | MRS + cys, 37° C., |
| Probiotic Capsules | | | | | | | | anaerobic |
| CVS Health Children's | TA244 | Feb. 1, 2021 | 545 | 0.32 | g | 50 | mL | MRS, 37° C., |
| Chewable Probiotic Tablets | | | | | | | | anaerobic |
| VSL#3 | 806084 | Jun. 1, 2020 | 441 | 0.55 | g | 50 | mL | MRS, 37° C., |
| | | | | | | | | anaerobic |
| TruBiotics Capsules | 55143 | May 1, 2020 | 318 | 0.18 | g | 50 | mL | MRS, 37° C., |
| | | | | | | | | anaerobic |
| Live Better Adult Advanced | SA004 | Feb. 1, 2020 | 227 | 0.5 | g | 50 | mL | MRS, 37° C., |
| Daily Probiotics | | | | | | | | anaerobic |
| Culturelle Digestive Daily | 19003CLG2 | Dec. 1, 2020 | 538 | 0.33 | g | 50 | mL | MRS, 37° C., |
| Probiotic Capsules | | | | | | | | anaerobic |
| Culturelle Kids Purely | 19051C3M11 | Nov. 1, 2021 | 865 | 1.5 | g | 50 | mL | MRS, 37° C., |
| Probiotics Packets | | | | | | | | anaerobic |

Details for FIGs. 6A-6B (high temperature stress test)

| | | | | | | | | |
|---|---|---|---|---|---|---|---|---|
| Mutaflor | 910230 | Feb. 4, 2020 | 197 | 0.18 | g | 50 | mL | LB, 37° C., |
| | | | | | | | | aerobic |
| Nitragin Gold Alfalfa and | NGA19123 | Jan. 31, 2022 | 929 | 0.05 | g | 4 | mL | TY, 30° C., |
| Sweet Clover | | | | | | | | aerobic |
| Florastor | 2049 | Aug. 1, 2021 | 741 | 0.29 | g | 50 | mL | YPD, 30° C., |
| | | | | | | | | aerobic |
| Schiff Digestive Advantage | 4345700 | Apr. 1, 2020 | 240 | 0.38 | g | 50 | mL | Nutrient, 37° C., |
| Prebiotic Fiber Plus | | | | | | | | aerobic |
| Probiotic Tablets | | | | | | | | |
| Schiff Digestive Advantage | 4167300 | May 1, 2020 | 269 | 0.53 | g | 50 | mL | Nutrient, 37° C., |
| Daily Probiotic Capsules | | | | | | | | aerobic |
| Align Probiotic Chewables | 83121453A2 | Aug. 1, 2020 | 368 | 0.19 | g | 50 | mL | MRS + cys, 37° C., |
| for Adults Supplement | | | | | | | | anaerobic |
| Align Probiotic Supplement | 83471453A1 | Sep. 1, 2020 | 419 | 0.19 | g | 50 | mL | MRS + cys, 37° C., |
| 24/7 Digestive Support | | | | | | | | anaerobic |
| Phillips' Colon Health | 8H17A | Aug. 1, 2020 | 359 | 0.35 | g | 50 | mL | MRS + cys, 37° C., |
| Probiotic Capsules | | | | | | | | anaerobic |
| CVS Health Children's | TA244 | Feb. 1, 2021 | 545 | 0.32 | g | 50 | mL | MRS, 37° C., |
| Chewable Probiotic Tablets | | | | | | | | anaerobic |
| VSL#3 | 806084 | Jun. 1, 2020 | 312 | 0.54 | g | 50 | mL | MRS, 37° C., |
| | | | | | | | | anaerobic |
| TruBiotics Capsules | 55143 | May 1, 2020 | 296 | 0.18 | g | 50 | mL | MRS, 37° C., |
| | | | | | | | | anaerobic |
| Live Better Adult Advanced | SA004 | Feb. 1, 2020 | 198 | 0.5 | g | 50 | mL | MRS, 37° C., |
| Daily Probiotics | | | | | | | | anaerobic |
| Culturelle Digestive Daily | 19003CLG2 | Dec. 1, 2020 | 502 | 0.32 | g | 50 | mL | MRS, 37° C., |
| Probiotic Capsules | | | | | | | | anaerobic |
| Culturelle Kids Purely | 19051C3M11 | Nov. 1, 2021 | 830 | 1.5 | g | 50 | mL | MRS, 37° C., |
| Probiotics Packets | | | | | | | | anaerobic |

“Days until exp.” is the number of days until the expiration date on the day the assessment was carried out. “Mass per dose” is the mean mass of the microbial fraction of the doses used in the assessment (see above). “Hyd. vol. per dose” is the volume used to rehydrate each dose. All culture media solidified with 1.5% agar (details are listed in Table 3).

TABLE 2
Library of materials used in stabilizer screen
“Solution” notes when materials were purchased or procured
as defined solutions and used as is. 1X concentrations were made
by volumetrically diluting 5X solutions 1:4 in ultrapure water.
Recipe for ATCC reagents at end of table.

| | Material | CAS or | | | 5X conc. |
|---|---|---|---|---|---|
| Index | name | product # | Vendor | Product # | (% w/w) |
| 1 | Bacto soytone | BD 243620 | BD | 243620 | 29 |
| 2 | ATCC Reagent 18 | ATCC recipe | — | — | solution |
| 3 | Palatinose hydrate | 343336-76-5 | Sigma | P2007 | 32 |
| 4 | Difco YPD Broth | BD 242820 | BD | 242820 | 29 |
| 5 | D-(+)-Turanose | 547-25-1 | Sigma | T2754 | 40 |
| 6 | maltitol | 585-88-6 | Sigma | M8892 | 44 |
| 7 | Difco Lactobacilli MRS Broth | BD 288130 | BD | 288130 | 29 |
| 8 | OPS MFDB | OPS 500-06 | OPS | 500-06 | solution |
| 9 | sucrose | 57-50-1 | Sigma | S7903 | 50 |
| 10 | Bacto beef extract, dessicated | BD 211520 | BD | 211520 | 29 |
| 11 | Melibiose | 585-99-9 | Sigma | M5500 | 44 |
| 12 | L-Rhamnose monohydrate | 10030-85-0 | Sigma | 83650 | 29 |
| 13 | Bacto tryptone | BD 211705 | BD | 211705 | 29 |
| 14 | D-(+)-Galactose | 59-23-4 | Sigma | G6404 | 40 |
| 15 | D-(+)-Melezitose monohydrate | 10030-67-8 | Sigma | 63620 | 25 |
| 16 | Bacto malt extract | BD 218630 | BD | 218630 | 39 |
| 17 | D(−)-Fructose | 57-48-7 | Acros | 161350010 | 24 |
| | | | Organics | | |
| 18 | ATCC Reagent 20 | ATCC recipe | — | — | solution |
| 19 | 1-Kestose | 470-69-9 | Sigma | 72555 | 7.4 |
| 20 | Maltodextrin 16.5-19.5 DE | 9050-36-6 | Sigma | 419699 | 44 |
| 21 | Glucose | 50-99-7 | Sigma | G8270 | 42 |
| 22 | 2-Deoxy-D-glucose | 154-17-6 | Sigma | D3179 | 23 |
| 23 | L-glutamic acid monosodium | 6106-04-3 | Sigma | 49621 | 44 |
| | salt monohydrate | | | | |
| 24 | Maltose monohydrate | 6363-53-7 | Sigma | M5885 | 3.9 |
| 25 | (+)-Sodium L-ascorbate | 134-03-2 | Sigma | A7631 | 21 |
| 26 | polydextrose | Honeyville | Honeyville | 77-121 | 50 |
| | | 77-121 | | | |
| 27 | D-(+)-Mannose | 3458-28-4 | Sigma | M6020 | 17 |
| 28 | D-(+)-Raffinose pentahydrate | 17629-30-0 | Sigma | 83400 | 40 |
| 29 | lactulose | 4618-18-2 | Sigma | 61360 | 38 |
| 30 | Gelysate peptone | BD 211870 | BD | 211870 | 29 |
| 31 | D-cellobiose | 528-50-7 | Sigma | C7252 | 6.3 |
| 32 | Sodium gluconate | 527-07-1 | Sigma | S2054 | 32 |
| 33 | Barium acetate | 543-80-6 | Sigma | 243671 | 32 |
| 34 | sodium citrate, dihydrate | 6132-04-3 | Mallinckrodt | 0754 | 33 |
| 35 | Trehalose dihydrate | 6138-23-4 | Sigma | T9531 | 3.9 |
| 36 | alpha-lactose, monohydrate | 5989-81-1 | Sigma | L2643 | 14 |
| 37 | Difco LB Broth, Lennox | BD 240230 | BD | 240230 | 29 |
| 38 | Difco M17 Broth | BD 218561 | BD | 218561 | 29 |
| 39 | Maltodextrin 4-7 DE | 9050-36-6 | Sigma | 419672 | 44 |
| 40 | 2-Phospho-L-ascorbic | 66170-10-3 | Sigma | 49752 | 33 |
| | acid trisodium salt | | | | |
| 41 | Difco Middlebrook 7H9 Broth | BD 271310 | BD | 271310 | 29 |
| 42 | chondroitin sulfate A | 39455-18-0 | Sigma | C8529 | 7.4 |
| 43 | Karaya Gum | 9000-36-6 | Sigma | G0503 | 1 |
| 44 | Bacto yeast extract | BD 212750 | BD | 212750 | 33 |
| 45 | Xylitol | 87-99-0 | Sigma | X3375 | 34 |
| 46 | beta-lactose | 5965-66-2 | Sigma | L3750 | 44 |
| 47 | Casamino acids | BD 228820 | BD | 228820 | 1.6 |
| 48 | Bacto Tryptic Soy Broth | BD 211825 | BD | 211825 | 29 |
| 49 | Skim milk powder | 999999-99-4 | sigma | 1153630500 | 29 |
| 50 | Potassium gluconate | 299-27-4 | Sigma | P1847 | 33 |
| 51 | D-Sorbitol | 50-70-4 | Sigma | S6021 | 55 |
| 52 | L-(+)-Arabinose | 5328-37-0 | Sigma | A3256 | 44 |
| 53 | Adonitol | 488-81-3 | Sigma | A5502 | 34 |
| 54 | Magnesium phosphate | 7782-75-4 | Sigma | 63080 | 30 |
| | dibasic trihydrate | | | | |
| 55 | Thymidine | 50-89-5 | Sigma | T9250 | 5.6 |
| 56 | sodium phosphate, monobasic | 7558-80-7 | Sigma | S8282 | 40 |
| 57 | γ-Aminobutyric acid | 56-12-2 | Sigma | A2129 | 50 |
| 58 | myo-Inositol | 87-89-8 | Sigma | I7508 | 9.9 |
| 59 | L-glutamic acid | 56-86-0 | Sigma | G8415 | 0.69 |
| 60 | Calcium D-gluconate | 299-28-5 | Sigma | C8231 | 2.5 |
| 61 | Potassium citrate | 6100-05-6 | Sigma | C3029 | 55 |
| | tribasic monohydrate | | | | |
| 62 | Lithium acetate dihydrate | 6108-17-4 | Sigma | L4158 | 40 |
| 63 | Cytidine | 65-46-3 | Sigma | C122106 | 7.4 |
| 64 | Difco Nutrient Broth | BD 234000 | BD | 234000 | 29 |
| 65 | Poly(vinylpolypyrrolidone) | 9003-39-8 | Sigma | 77627 | 7.4 |
| 66 | L-Histidine | 71-00-1 | Sigma | H8000 | 3.3 |
| 67 | Magnesium D-gluconate hydrate | 3632-91-5 | Sigma | M7554 | 9.1 |
| 68 | Aluminum silicate | Sigma 520179 | Sigma | 520179 | 40 |
| 69 | Lecithin, Refined | 8002-43-5 | Alfa Aesar | 36486 | 15 |
| 70 | Celite ® 545 | Sigma CX0574-1 | Sigma | CX0574-1 | 11 |
| 71 | 1,4-Piperazinediethanesulfonic acid | 5625-37-6 | Sigma | P1851 | 10 |
| 72 | Amygdalin | 29883-15-6 | Sigma | 10050 | 7.4 |
| 73 | Catalase from bovine liver | 9001-05-2 | Sigma | C9322 | 0.07 |
| 74 | Ammonium sulfate | 7783-20-2 | Mallinckrodt | 3512 | 38 |
| 75 | OPS Lyophilization reagent | OPS 500-02 | OPS | 500-02 | solution |
| 76 | α-Cyclodextrin | 10016-20-3 | Sigma | C4642 | 0.79 |
| 77 | Lithium chloride | 7447-41-8 | Sigma | 62478 | 35 |
| 78 | D-glucosamine HCl | 66-84-2 | Sigma | G1514 | 7.4 |
| 79 | Catalase (Aspergillus niger) | 9001-05-2 | Sigma | C3515 | 0.07 |
| 80 | N,N-Bis(2-hydroxyethyl)-2- | 10191-18-1 | Sigma | B9879 | 50 |
| | aminoethanesulfonic acid | | | | |
| 81 | Yeast nitrogen base | MP 4027512 | MP biomedicals | 4027512 | 9.1 |
| 82 | Adenosine | 58-61-7 | Sigma | A9251 | 0.53 |
| 83 | Hypoxanthine | 68-94-0 | Sigma | H9636 | 0.06 |
| 84 | 1-Adamantylamine | 768-94-5 | Sigma | 138576 | 0.5 |
| 85 | Mucin (Porcine, type III) | 84082-64-4 | Sigma | M1778 | 0.4 |
| 86 | Allantoin | 97-59-6 | Sigma | 05670 | 0.42 |
| 87 | 3,4-dihydroxy-DL-phenylalanine | 63-84-3 | Sigma | D9503 | 0.4 |
| 88 | hydrocortisone | 50-23-7 | Sigma | H4001 | 0.02 |
| 89 | Hydroxyapatite nanopowde | 12167-74-7 | Sigma | 677418 | 23 |
| 90 | Sodium caseinate | 9005-46-3 | Sigma | C8654 | 3.9 |
| 91 | O-Phospho-DL-serine | 17885-08-4 | Sigma | 79710 | 1.5 |
| 92 | N-Phenylthiourea | 103-85-5 | Sigma | P7629 | 0.2 |
| 93 | N-Hydroxyphthalimide | 524-38-9 | Sigma | H53704 | 0.25 |
| 94 | L-Tyrosine | 60-18-4 | Sigma | T3754 | 0.03 |
| 95 | 1-Pentanol | 71-41-0 | Sigma | 138975 | 2.2 |
| 96 | sodium acetate, anhydrous | 127-09-3 | Mallinckrodt | 7372 | 27 |
| 97 | Ethylenediaminetetraacetic acid | 60-00-4 | Sigma | 03690 | solution |
| 98 | 2,2-Bis(hydroxymethyl)propionic acid | 4767-03-7 | Sigma | 106615 | 9.1 |
| 99 | Pimelic acid | 111-16-0 | Sigma | P45001 | 3.9 |
| 100 | 2,4-Pentanediol | 625-69-4 | Sigma | 156019 | 50 |
| 101 | 1-Thioglycerol | 96-27-5 | Sigma | M6145 | 50 |
| 102 | 1,5,7-Triazabicyclo[4.4.0]dec-5-ene | 5807-14-7 | Sigma | 345571 | 9.1 |
| 103 | L-Arginine | 74-79-3 | Sigma | A5006 | 13 |
| 104 | Choline bitartrate | 87-67-2 | Sigma | C1629 | 7.4 |
| 105 | Bacto peptone | BD 211677 | BD | 211677 | 29 |
| 106 | L-serine | 56-45-1 | Sigma | S4500 | 3.9 |
| 107 | Meglumine | 6284-40-8 | Sigma | M9179 | 44 |
| 108 | L-Threonine | 72-19-5 | Sigma | T8625 | 7.2 |
| 109 | Tris(hydroxymethyl)aminomethane | 77-86-1 | Sigma | T6791 | 31 |
| 110 | erythritol | 149-32-6 | Sigma | E7500 | 33 |
| 111 | 2-Ethyl-1-hexanol | 104-76-7 | Sigma | 04050 | 0.07 |
| 112 | uracil | 66-22-8 | MP biomedicals | 4061212 | 0.29 |
| 113 | caffeine | 58-08-2 | Sigma | C0750 | 1.7 |
| 114 | ricinoleic acid | 141-22-0 | Sigma | 83903 | 0.28 |
| 115 | Suberic acid | 505-48-6 | Sigma | S5200 | 0.13 |
| 116 | Neohesperidin dihydrochalcone | 20702-77-6 | Sigma | N8757 | 0.08 |
| 117 | D-(+)-Xylose | 58-86-6 | Sigma | 95729 | 50 |
| 118 | Calcium acetate hydrate | 114460-21-8 | Sigma | C1000 | 14 |
| 119 | 6-Aminocaproic acid | 60-32-2 | Sigma | A2504 | 29 |
| 120 | Sucrose octaacetate | 126-14-7 | Sigma | W303801 | 0.06 |
| 121 | α-Cyano-4-hydroxycinnamic acid | 28166-41-8 | Sigma | 55341 | 0.48 |
| 122 | trans-cinnamic acid | 140-10-3 | Sigma | C80857 | 0.03 |
| 123 | beta-cyclodextrin | 7585-39-9 | Sigma | C4767 | 1.5 |
| 124 | N-Hydroxysuccinimide | 6066-82-6 | Sigma | 130672 | 3.9 |
| 125 | Sulfanilamide | 63-74-1 | Sigma | S9251 | 0.67 |
| 126 | boric acid | 10043-35-3 | Sigma | B6768 | 3.1 |
| 127 | Triacetin | 102-76-1 | Sigma | 525073 | 5.8 |
| 128 | Mannitol | 69-65-8 | Sigma | M4125 | 12 |
| 129 | Propyl gallate | 121-79-9 | Sigma | P3130 | 0.28 |
| 130 | Betaine | 107-43-7 | Sigma | B2629 | 57 |
| 131 | D-tyrosine | 556-02-5 | Fluka | 93840 | 0.04 |
| 132 | (−)-Terpinen-4-ol | 20126-76-5 | Sigma | 11584 | 10 |
| 133 | L-LEUCINE | 61-90-5 | MP biomedicals | 4060512 | 1.9 |
| 134 | fumaric acid | 110-17-8 | Sigma | F8509 | 0.5 |
| 135 | L-Methionine | 63-68-3 | Sigma | M9625 | 4.3 |
| 136 | Pentaerythritol | 115-77-5 | Sigma | P4755 | 5.5 |
| 137 | Zinc acetate | 557-34-6 | Sigma | 383317 | 20 |
| 138 | Tyramine | 51-67-2 | Sigma | T90344 | 0.83 |
| 139 | 4-Guanidinobutyric acid | 463-00-3 | Sigma | G6503 | 7.4 |
| 140 | Guanosine 5′-monophosphate | 5550-12-9 | Sigma | G8377 | 3.9 |
| | disodium salt hydrate | | | | |
| 141 | Taurine | 107-35-7 | Sigma | T0625 | 7.1 |
| 142 | Albumin, human | 70024-90-7 | Sigma | A1653 | 7.4 |
| 143 | β-Alanine | 107-95-9 | Sigma | 146064 | 31 |
| 144 | Saccharin | 81-07-2 | Sigma | 109185 | 0.27 |
| 145 | salicylic acid | 69-72-7 | Sigma | 247588 | 0.18 |
| 146 | 1,4-Cyclohexanedimethanol | 105-08-8 | Sigma | 125598 | 43 |
| 147 | L-alanine | 56-41-7 | sigma | A7627 | 12 |
| 148 | Ammonium bromide | 12124-97-9 | Fluka | 213349 | 39 |
| 149 | trans-4-Hydroxy-L-proline | 51-35-4 | Sigma | H54409 | 22 |
| 150 | Propyl 4-hydroxybenzoate | 94-13-3 | Sigma | P53357 | 0.03 |
| 151 | 1,4-Benzenedimethanol | 589-29-7 | Sigma | B3000 | 2.3 |
| 152 | O-tert-Butyl-L-serine | 18822-58-7 | Sigma | B6278 | 9.1 |
| 153 | Acetylsalicylic acid | 50-78-2 | Sigma | 23963 | 0.22 |
| 154 | Urea | 57-13-6 | Sigma | U4884 | 44 |
| 155 | L-Tryptophan | 73-22-3 | Sigma | T0254 | 0.9 |
| 156 | 3-Pentanol | 584-02-1 | Sigma | P8025 | 4.2 |
| 157 | 3-Guanidinopropionic acid | 353-09-3 | Sigma | G6878 | 7.4 |
| 158 | dioctyl-sulfosuccinate | 577-11-7 | Sigma | 323586 | 1.2 |
| 159 | Octanoic acid | 124-07-2 | Sigma | C2875 | 0.05 |
| 160 | Adipic acid | 124-04-9 | Sigma | 09582 | 1.8 |
| 161 | Triglycerol | 20411-31-8 | Sigma | 17782 | 33 |
| 162 | sodium triphosphate, pentabasic | 7758-29-4 | Sigma | T5883 | 14 |
| 163 | Tris(hydroxymethyl)aminomethane | 1185-53-1 | Sigma | T6666 | 31 |
| | hydrochloride | | | | |
| 164 | L-Proline | 147-85-3 | Sigma | P0380 | 57 |
| 165 | Choline chloride | 67-48-1 | Sigma | C7527 | 29 |
| 166 | L-valine | 72-18-4 | Sigma | V0500 | 4.4 |
| 167 | Potassium pyrophosphate | 7320-34-5 | Sigma | 322431 | 60 |
| 168 | Sodium dodecyl sulfate | 151-21-3 | Sigma | L3771 | 7.4 |
| 169 | β-Glycerophosphate | 154804-51-0 | Sigma | G9422 | 3.9 |
| | disodium salt hydrate | | | | |
| 170 | hydroquinone | 123-31-9 | Sigma | H9003 | 6.6 |
| 171 | N,N-Bis(2-hydroxyethyl)ethylenediamine | 3197-06-6 | Sigma | 480614 | 9.1 |
| 172 | 3-Methyl-1,5-pentanediol | 4457-71-0 | Sigma | 68346 | 50 |
| 173 | Sodium bicarbonate | 144-55-8 | Sigma | S6014 | 7.4 |
| 174 | sodium sulfate | 7757-82-6 | Sigma | 238597 | 18 |
| 175 | L-ascorbic acid | 50-81-7 | Sigma | A5960 | 21 |
| 176 | Aluminum L-lactate | 18917-91-4 | Sigma | 430633 | 9.1 |
| 177 | Sodium chloride | 7647-14-5 | Sigma | S7653 | 22 |
| 178 | D-(−)-Salicin | 138-52-3 | Sigma | S0625 | 3.4 |
| 179 | 1,2,4,5-Benzenetetracarboxylic acid | 89-05-4 | Sigma | B4007 | 1.1 |
| 180 | 3,6-Dimethyl-1,4-dioxane-2,5-dione | 95-96-5 | Sigma | 303143 | 0.99 |
| 181 | (±)-1,3-Butanediol | 107-88-0 | Sigma | 18940 | 50 |
| 182 | Sucralose | 56038-13-2 | Sigma | 69293 | 7.4 |
| 183 | Calcium L-lactate hydrate | 41372-22-9 | Sigma | L4388 | 14 |
| 184 | Potassium Chloride | 7447-40-7 | Mallinckrodt | 6858 | 22 |
| 185 | sodium sulfite | 7757-83-7 | Sigma | 71991 | 20 |
| 186 | sodium phosphate, dibasic | 7558-79-4 | Mallinckrodt | 7917 | 9.1 |
| 187 | Ammonium carbonate | 506-87-6 | Sigma | 20786 | 17 |
| 188 | Bentonite | 1302-78-9 | Sigma | 285234 | 1.6 |
| 189 | Taurocholic acid | 345909-26-4 | Sigma | T4009 | 7.4 |
| | sodium salt hydrate | | | | |
| 190 | Carbon, mesoporous | 1333-86-4 | Sigma | 699624 | 2.6 |
| 191 | 4,7,10-Trioxa-1,13-tridecanediamine | 4246-51-9 | Sigma | 369519 | 9.1 |
| 192 | Potassium Phosphate, monobasic | 7778-77-0 | Mallinckrodt | 7100 | 15 |
| 193 | Fructooligosaccharides | Sigma F8052 | Sigma | F8052 | 15 |
| 194 | Copper(II) sulfate | 7758-98-7 | Sigma | C1297 | 14 |
| 195 | Sodium carbonate | 497-19-8 | Sigma | 223484 | 19 |
| 196 | 3-(Diethylamino)-1,2-propanediol | 621-56-7 | Sigma | 210226 | 9.1 |
| 197 | D-(−)-Ribose | 50-69-1 | Sigma | R7500 | 40 |
| 198 | N-(1-Naphthyl)ethylenediamine | 1465-25-4 | Sigma | 222488 | 0.16 |
| | dihydrochloride | | | | |
| 199 | Potassium acetate | 127-08-2 | Sigma | 236497 | 62 |
| 200 | L-lysine | 56-87-1 | Sigma | L5501 | 44 |
| 201 | Manganese(II) chloride | 13446-34-9 | Sigma | M8054 | 47 |
| | tetrahydrate | | | | |
| 202 | DAB-Am-4 | 120239-63-6 | Sigma | 460699 | 9.1 |
| 203 | ammonium acetate | 631-61-8 | Mallinckrodt | 3272 | 44 |
| 204 | N,N′-Dimethylethylenediamine | 110-70-3 | Sigma | D157805 | 9.1 |
| 205 | citric acid, monohydrate | 5949-29-1 | Mallinckrodt | 0627 | 32 |
| 206 | 5-Aminovaleric acid | 660-88-8 | Sigma | 123188 | 44 |
| 207 | Citric acid | 77-92-9 | Sigma | 251275 | 32 |
| 208 | Guanidine hydrochloride | 50-01-1 | Sigma | G4505 | 63 |
| 209 | octyl-beta-D-glucopyranoside | 29836-26-8 | Sigma | O8001 | 7.4 |
| 210 | DL-β-(2-Thienyl)serine | 32595-59-8 | Sigma | T5000 | 0.99 |
| 211 | Cystamine dihydrochloride | 56-17-7 | Sigma | C8707 | 33 |
| 212 | mercaptosuccinic acid | 70-49-5 | Sigma | 88460 | 24 |
| 213 | Glycine | 56-40-6 | Sigma | G7126 | 17 |
| 214 | glycolic acid | 79-14-1 | Sigma | 124737 | 44 |
| 215 | Glycerol phosphate calcium salt | 58409-70-4 | Sigma | G6626 | 3.9 |
| 216 | potassium carbonate | 584-08-7 | Sigma | P5833 | 47 |
| 217 | 3-Methyl-1,3-butanediol | 2568-33-4 | Sigma | 65965 | 50 |
| 218 | cysteamine | 60-23-1 | Sigma | 30070 | 33 |
| 219 | Pyridoxine hydrochloride | 58-56-0 | Sigma | P6280 | 15 |
| 220 | L-(+)-Lactic acid | 79-33-4 | Sigma | L1750 | 7.4 |
| 221 | itaconic acid | 97-65-4 | Sigma | I29204 | 6.3 |
| 222 | Potassium hydroxide | 1310-58-3 | Sigma | P5958 | 29 |
| 223 | O-Acetyl-L-serine hydrochloride | 66638-22-0 | Sigma | A6262 | 9.1 |
| 224 | Sodium hydroxide | 1310-73-2 | Mallinckrodt | 7708 | 42 |
| 225 | 5-Nitro-m-xylene-α,α′-diol | 71176-55-1 | Sigma | 184799 | 1.5 |
| 226 | 1,7-Heptanediol | 629-30-1 | Sigma | H2201 | 40 |
| 227 | L-(+)-Tartaric acid | 87-69-4 | Sigma | T1807 | 52 |
| 228 | N,N′-Dimethyl-1,3-propanediamine | 111-33-1 | Sigma | 308110 | 9.1 |
| 229 | 3,3′-Thiodipropionic acid | 111-17-1 | Sigma | T30201 | 2.9 |
| 230 | 1,4-Dimethylpyridinium | 78105-28-9 | Sigma | 514888 | 9.1 |
| | p-toluenesulfonate | | | | |
| 231 | sodium nitrite | 7632-00-0 | Sigma | S2252 | 40 |
| 232 | Triethyl citrate | 77-93-0 | Sigma | 27500 | 4.9 |
| 233 | 2-Methyl-1-propanol | 78-83-1 | Sigma | 294829 | 6.4 |
| 234 | Potassium iodide | 7681-11-0 | Fluka | 60399 | 52 |
| 235 | DL-Dithiothreitol | 3483-12-3 | Sigma | D5545 | 40 |
| 236 | DL-tartaric acid | 133-37-9 | Sigma | T400 | 14 |
| 237 | sodium cholate, hydrate | 206986-87-0 | Sigma | 270911 | 33 |
| 238 | sodium metabisulfite | 7681-57-4 | Sigma | S9000 | 35 |
| 239 | Sodium taurodeoxycholate hydrate | 207737-97-1 | Sigma | T0875 | 7.4 |
| 240 | Sodium iodide | 7681-82-5 | Sigma | 217638 | 62 |
| 241 | 1,4-Pentanediol | 626-95-9 | Sigma | 194182 | 50 |
| 242 | 2-Amino-5-diethylaminopentane | 140-80-7 | Sigma | A48806 | 9.1 |
| 243 | 2,2-Dimethyl-1,3-propanediol | 126-30-7 | Sigma | 538256 | 40 |
| 244 | Ethanolamine | 141-43-5 | Sigma | E0135 | 44 |
| 245 | trans-aconitic acid | 4023-65-8 | Sigma | 01600 | 24 |
| 246 | (±)-1,2,4-Butanetriol | 3068-00-6 | Sigma | 19040 | 50 |
| 247 | Aluminum sulfate hydrate | 7784-31-8 | Sigma | 227617 | 44 |
| 248 | 3-Piperidino-1,2-propanediol | 4847-93-2 | Sigma | 218499 | 9.1 |
| 249 | 1,3-Benzenedimethanol | 626-18-6 | Sigma | 196533 | 9.1 |
| 250 | 8-Aminooctanoic acid | 1002-57-9 | Sigma | 855294 | 20 |
| 251 | Magnesium chloride hexahydrate | 7791-18-6 | Sigma | M2670 | 33 |
| 252 | Calcium chloride dihydrate | 10035-04-8 | Sigma | C5080 | 11 |
| 253 | malic acid | 6915-15-7 | Sigma | M8304 | 31 |
| 254 | L-Cysteine hydrochloride | 7048-04-6 | Sigma | C7880 | 3.9 |
| | monohydrate | | | | |
| 255 | Zinc sulfate monohydrate | 7446-19-7 | Sigma | 307491 | 20 |
| 256 | 1,2,6-Hexanetriol | 106-69-4 | Sigma | T66206 | 50 |
| 257 | 1,3-Cyclopentanediol, | 59719-74-3 | Sigma | 192805 | 50 |
| | mixture of cis and trans | | | | |
| 258 | Camphor-10-sulfonic acid (β) | 5872-08-2 | Sigma | 147923 | 33 |
| 259 | succinic acid | 110-15-6 | Sigma | S9512 | 6.3 |
| 260 | Butylated hydroxyanisole | 25013-16-5 | Sigma | B1253 | 0.06 |

ATCC reagent 18:
0.75 g Trypticase Soy Broth
10 g Sucrose
5 g Bovine Serum Albumin Fraction V
100 mL Distilled water
Filter-sterilize through a 0.2 μm filter.
ATCC reagent 20:
20 g Sucrose
10 g Bovine Serum Albumin Fraction V
100 mL Distilled water
Filter-sterilize through a 0.2 μm filter.

TABLE 3
Growth mediums used in the Examples.
When a recipe is listed, details of the individual subcomponents
are listed at end of table. Media used as a solid for plating
assays was always solidified with 1.5% w/v agar.

| | | | |
|---|---|---|---|
| Short name | Name or recipe | Vendor | Product # |

Final culture media used

| Agar | Bacto Agar | BD | 214010 |
|---|---|---|---|
| LB | Difco LB Broth, Lennox | BD | 240230 |
| YPD | Difco YPD Broth | BD | 242820 |
| Nutrient | Difco Nutrient Broth | BD | 234000 |
| MRS | Difco Lactobacilli MRS | BD | 288130 |
| MRS + cys | MRS + 0.05% w/v | | |
| TY | per liter: 6 g Tryptone, 3 g Yeast | | |
| 1/2X BNM | | per liter: 0.172 g CaSO4, 0.195 g MES, | |
| | | 2.5 mL NOD major, 2.5 mL NOD minor 1, 2.5 mL | |
| | pH to 6.5 with 2N KOH | | |

Subcomponents referenced in 1/2X BNM

| | |
|---|---|
| NOD major | per liter: 24.4 g MgSO4, 13.6 g KH2PO4 |
| NOD minor 1 | per liter: 0.92 g ZnSO4, 0.62 g H3BO3, 1.69 g MnSO4 |
| NOD minor 2 | per liter: 0.05 g Na2MoO4, 0.0032 g CuSO4, 0.005 |
| Fe-EDTA | per liter: 3.73 g Na2EDTA, 2.78 g FeSO4 |

Individual components referenced above

| | | | |
|---|---|---|---|
| Tryptone | Bacto Tryptone | BD | 211705 |
| Yeast extract | Bacto Yeast Extract | BD | 212750 |
| CaCl2 | Calcium chloride | Sigma | C5080 |
| Cysteine | L-Cysteine | Sigma | C7880 |
| CaSO4 | Calcium sulfate | Sigma | C3771 |
| MES | MES hemisodium salt | Sigma | M8902 |
| MgSO4 | Magnesium sulfate | Sigma | 63138 |
| KH2PO4 | Potassium phosphate | Sigma | P5655 |
| ZnSO4 | Zinc sulfate | Sigma | Z1001 |
| H3BO3 | Boric acid | Sigma | B0394 |
| MnSO4 | Manganese (II) sulfate | Sigma | M7899 |
| Na2MoO4 | Sodium molybdate | Sigma | M1651 |
| CuSO4 | Copper (II) sulfate | Sigma | C1297 |
| CoCl2 | Cobalt (II) chloride | Sigma | C2911 |
| Na2EDTA | EDTA disodium salt | Sigma | E6635 |
| FeSO4 | Iron (II) sulfate | Sigma | F8263 |
| KOH | Potassium hydroxide | Sigma | P5958 |

TABLE 4
Strains and plasmids used in the Examples.

| Strain | Source or Genotype | Note |
|---|---|---|
| Escherichia coli | Isolated from | Used in FIGs. 2A-2E, FIGs. 7A-7C, |
| Nissle 1917 | Mutaflor | FIG. 8, FIG. 12, FIG. 13, FIG. 15 |
| Saccharomyces | Isolated from | Used in FIGs. 2A-2E, FIGs. 7A-7C, |
| boulardii | Florastor | FIG. 8 |
| Ensifer meliloti | ATCC 51124 | Used in FIGs. 2A-2E, FIGs. 4A-4H, |
| Rm1021 | | FIGs. 7A-7C, FIG. 8, FIG. 18 |
| Lactobacillus | CCUG 61730 | Used in FIGs. 2A-2E, FIGs. 7A-7C, |
| plantarum NC8 | | FIG. 8 |
| Shigella fexneri 2457T | ATCC 700930 | Used in FIGs. 4A-4H, FIGs. 20A-20B |
| Escherichia coli DH5a | ThermoFisher | Used in FIGs. 4A-4H, FIGs. 20A-20B |
| Medicago truncatula | Noble Research | Used in FIGs. 4A-4H |
| A17 | Institute | |
| sMJ026 | E. coli Nissle | Used in all other figures referencing |
| | 1917 + pAKlux2 | E. coli Nissle 1917 |
| pAKlux2 (plasmid | Addgene #14080 | Source of plasmid for sMJ026 |
| strain) | | |

### REFERENCES

1. Vitorino, L. C. & Bessa, L. A. Technological Microbiology: Development and Applications. Front. Microbiol. 8, (2017).
2. Ozdemir, T., Fedorec, A. J. H., Danino, T. & Barnes, C. P. Synthetic Biology and Engineered Live Biotherapeutics: Toward Increasing System Complexity. Cell Syst. 7, 5-16 (2018).
3. Arif, I., Batool, M. & Schenk, P. M. Plant Microbiome Engineering: Expected Benefits for Improved Crop Growth and Resilience. Trends Biotechnol. 0, (2020).
4. Menezes Amor A., Cumbers John, Hogan John A., & Arkin Adam P. Towards synthetic biological approaches to resource utilization on space missions. J. R. Soc. Interface 12, 20140715 (2015).
5. Jimenez, M., Langer, R. & Traverso, G. Microbial therapeutics: New opportunities for drug delivery. J. Exp. Med. 216, 1005-1009 (2019).
6. Hyronimus, B., Le Marrec, C., Hadj Sassi, A. & Deschamps, A. Acid and bile tolerance of spore-forming lactic acid bacteria. Int. J. Food Microbiol. 61, 193-197 (2000).
7. Kurtz Caroline, Denney William S., Blankstein Larry, Guilmain Sarah E., Machinani Suman, Kotula Jonathan, Saha Saurabh, Miller Paul, & Brennan Aoife M. Translational Development of Microbiome-Based Therapeutics: Kinetics of E. coli Nissle and Engineered Strains in Humans and Nonhuman Primates. Clin. Transl. Sci. 0, (2017).
8. Gurbatri, C. R., Lia, I., Vincent, R., Coker, C., Castro, S., Treuting, P. M., Hinchliffe, T. E., Arpaia, N. & Danino, T. Engineered probiotics for local tumor delivery of checkpoint blockade nanobodies. Sci. Transl. Med. 12, eaax0876 (2020).
9. Remington, J. P. & Adejare, A. Remington, the science and practice of pharmacy. (Academic press, 2021).
10. Prakash, O., Nimonkar, Y. & Shouche, Y. S. Practice and prospects of microbial preservation. FEMS Microbiol. Lett. 339, 1-9 (2013).
11. Doses in Our Daily Lives. NRC Web nrc.gov/about-nrc/radiation/around-us/doses-daily-lives.html.
12. Zhang, S., Wimmer-Schweingruber, R. F., Yu, J., Wang, C., Fu, Q., Zou, Y., Sun, Y., Wang, C., Hou, D., Böttcher, S. I., Burmeister, S., Seimetz, L., Schuster, B., Knierim, V., Shen, G., Yuan, B., Lohf, H., Guo, J., Xu, Z., Freiherr von Forstner, J. L., Kulkarni, S. R., Xu, H., Xue, C., Li, J., Zhang, Z., Zhang, H., Berger, T., Matthiä, D., Hellweg, C. E., Hou, X., Cao, J., Chang, Z., Zhang, B., Chen, Y., Geng, H. & Quan, Z. First measurements of the radiation dose on the lunar surface. Sci. Adv. 6, eaaz1334 (2020).
13. Zeitlin, C., Cleghorn, T., Cucinotta, F., Saganti, P., Andersen, V., Lee, K., Pinsky, L., Atwell, W., Turner, R. & Badhwar, G. Overview of the Martian radiation environment experiment. Adv. Space Res. 33, 2204-2210 (2004).
14. Hassler, D. M. et al. Mars' Surface Radiation Environment Measured with the Mars Science Laboratory's Curiosity Rover. Science 343, 1244797 (2014).
15. Dachev, T. P., Bankov, N. G., Tomov, B. T., Matviichuk, Yu. N., Dimitrov, P1. G., Häder, D.-P. & Horneck, G. Overview of the ISS Radiation Environment Observed during the ESA EXPOSE-R2 Mission in 2014-2016. Space Weather 15, 1475-1489 (2017).
16. Lambert, P. A. Radiation Sterilization, in Russell, Hugo & Ayliffe's 294-305 (John Wiley & Sons, Ltd, 2013). doi:10.1002/9781118425831.ch15b.
17. Ivanova, E. P. & Skalozub, O. M. The effectiveness of the use of Sinorhizobium meliloti in the cultivation of variable alfalfa (Medicago varia Mart.). IOP Conf. Ser. Earth Environ. Sci. 962, 012017 (2022).
18. Milo, R., Jorgensen, P., Moran, U., Weber, G. & Springer, M. BioNumbers—the database of key numbers in molecular and cell biology. Nucleic Acids Res. 38, D750-D753 (2010).
19. Abràmoff, M. D., Magalhães, P. J. & Ram, S. J. Image processing with ImageJ. Biophotonics Int. 11, 36-42 (2004).
20. Sugawara, M. & Sadowsky, M. J. Enhanced Nodulation and Nodule Development by nolR Mutants of Sinorhizobium medicae on Specific Medicago Host Genotypes. Mol. Plant-Microbe Interactions® 27, 328-335 (2014).
21. Barker, D., Pfaff, T., Moreau, D., Groves, E., Ruffel, S., Lepetit, M., Whitehand, S., Maillet, F., Nair, R. & Journet, E.-P. Growing M. truncatula: choice of substrates and growth conditions. (The samuel Roberts Noble Fondation, 2006).
22. Protocol for nodulation assay in pouches. Legume-Microbe Interactions Laboratory staceylab.missouri.edu/protocol-nodulation-assay-pouches/(2016).
23. Rowlett, V. W. et al. Impact of Membrane Phospholipid Alterations in Escherichia coli on Cellular Function and Bacterial Stress Adaptation. J. Bacteriol. 199, e00849-16 (2017).
24. Bogdanov, M., Dowhan, W. & Vitrac, H. Lipids and topological rules governing membrane protein assembly. Biochim. Biophys. Acta BBA—Mol. Cell Res. 1843, 1475-1488 (2014).
25. Sonnenborn, U. & Schulze, J. The non-pathogenic Escherichia coli strain Nissle 1917—features of a versatile probiotic. Microb. Ecol. Health Dis. 21, 122-158 (2009).
26. Connor, T. R. et al. Species-wide whole genome sequencing reveals historical global spread and recent local persistence in Shigella flexneri. eLife 4, e07335 (2015).
27. Long, S. R. Rhizobium-legume nodulation: Life together in the underground. Cell 56, 203-214 (1989).
28. Deaker, R. Legume seed inoculation technology? a review. Soil Biol. Biochem. 36, 1275-1288 (2004).
29. Pedrini, S., Merritt, D. J., Stevens, J. & Dixon, K. Seed Coating: Science or Marketing Spin?Trends Plant Sci. 22, 106-116 (2017).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

1. A composition comprising Escherichia coli (E. coli) and a first stabilizing excipient selected from an enzymatic digest of soy, palatinose hydrate, D-(+)-turanose, maltitol, potassium gluconate, melibiose, sucrose, an animal origin peptone derived from an infusion of beef L-rhamnose monohydrate, (+)-Sodium L-ascorbate, an animal origin pancreatic digest of casein trehalose dihydrate, D-(+)-galactose, a water-soluble portion of malted barley, D-(+)-melezitose monohydrate, beta-lactose, D(−)-fructose, glucose, and 1-kestose, or a combination thereof.

2-5. (canceled)

6. The composition of claim 1, further comprising a second stabilizing excipient selected from short inulin, concentrate of the water-soluble portion of autolyzed Saccharomyces cerevisiae cells, animal origin pancreatic digest of casein, porcine mucin type III, 1,4-benzenedimethanol, caffeine, enzymatic digest of soy, sucralose, dioctyl-sulfosuccinate, 2-methyl-1-propanol, propyl gallate, β-glycerophosphate disodium salt hydrate, DL-β-(2-thienyl)serine, melibiose, animal origin peptone derived from an infusion of beef, sodium citrate dihydrate, N-phenylthiourea, 4-guanidinobutyric acid, calcium D-gluconate, or a combination thereof.

7-8. (canceled)

9. The composition of claim 6, wherein the first stabilizing excipient is melibiose, and the second stabilizing excipient is concentrate of the water-soluble portion of autolyzed Saccharomyces cerevisiae cells or caffeine.

10-16. (canceled)

17. The composition of claim 1, wherein the E. coli is E. coli Nissle 1917.

18. A composition comprising Saccharomyces boulardii (S. boulardii) and a first stabilizing excipient selected from L-glutamic acid monosodium salt monohydrate, skim milk powder. D-(+)-turanose, water-soluble portion of malted barley, maltitol, melibiose, lactulose, D-(+)-raffinose pentahydrate, palatinose hydrate, sucrose, animal origin pancreatic digest of casein, glucose, enzymatic digest of soy, potassium gluconate, polydextrose, sodium gluconate, or a combination thereof.

19-20. (canceled)

21. A composition comprising Lactobacillus plantarum (L. plantarum) and a first stabilizing excipient selected from pancreatic digest of gelatin, water-soluble portion of malted barley, animal origin pancreatic digest of casein, sucrose, animal origin peptone derived from an infusion of beef, D-sorbitol, polydextrose, trehalose dihydrate, L-glutamic acid monosodium salt monohydrate, beta-lactose, maltodextrin, L-rhamnose monohydrate, 1-kestose, animal-origin, enzymatic digest of bovine and porcine animal proteins, chondroitin sulfate A, or a combination thereof.

22-23. (canceled)

24. A composition comprising Ensifer meliloti (E. meliloti) and a first stabilizing excipient selected from enzymatic digest of soy skim milk powder, polydextrose, water-soluble portion of malted barley L-glutamic acid monosodium salt monohydrate, maltodextrin, palatinose hydrate, D-(+)-melezitose monohydrate, trehalose dihydrate, 1-kestose, maltose monohydrate, alpha-lactose monohydrate, sucrose, D-cellobiose, D-(+)-raffinose pentahydrate, melibiose, or a combination thereof.

25-31. (canceled)

32. The composition of claim 1, wherein the composition exhibits at least about 1×10⁶, at least about 1×10⁷, at least about 1×10⁸, at least about 1×10⁹, or at least about 1×10¹⁰ colony forming units (CFU)/gram after 30 days at 23° C.

33. (canceled)

34. The composition of claim 1, wherein the composition is stable at about 23° C., about 37° C., or about 50° C. for at least 1 month.

35. (canceled)

36. The composition of claim 1, wherein the composition is stable at less than about 5% relative humidity for at least 1 month.

37-39. (canceled)

40. The composition of claim 1, wherein the composition is stable upon exposure to an organic solvent.

41-43. (canceled)

44. The composition of claim 1, wherein the composition retains at least about 5%, at least about 10%, or at least about 15% viability upon tableting.

45-62. (canceled)

63. The composition of claim 1, wherein the composition is stable upon exposure to ionizing radiation.

64-79. (canceled)

80. A method of delivering E. coli to a subject in need thereof, comprising administering to the subject the composition of claim 1.

81. A method of delivering S. boulardii to a subject in need thereof, comprising administering to the subject the composition of claim 18.

82. A method of delivering L. plantarum to a subject in need thereof, comprising administering to the subject the composition of claim 21.

83. A method of delivering E. meliloti to a subject in need thereof, comprising administering to the subject the composition of claim 24.

84. A method of inducing bacterial growth in a subject or in a cell, tissue, or biological sample, comprising administering to the subject or contacting the cell, tissue, or biological sample with the composition of claim 1.

85. (canceled)

86. (canceled)

87. A method of inhibiting an enteric pathogen in a subject or in a cell, tissue, or biological sample, comprising administering to the subject or contacting the cell, tissue, or biological sample with the composition of claim 1.

88. (canceled)

89. A method of treating dysbiosis in a subject in need thereof, comprising administering to the subject the composition of claim 1.

90. (canceled)
